(19) United States
(12) Patent Application Publication
(10) Pub. No.: US 2025/0299695 A1
(43) Pub. Date: Sep. 25, 2025

(54) FLUORINE-CONTAINING ETHER COMPOUND, LUBRICANT FOR MAGNETIC RECORDING MEDIUM, AND MAGNETIC RECORDING MEDIUM

(71) Applicant: Resonac Corporation, Tokyo (JP)

(72) Inventors: Kohei SAGA, Tokyo (JP); Daisuke YAGYU, Tokyo (JP); Takuya UNO, Tokyo (JP); Yutaka TANJI, Tokyo (JP)

(73) Assignee: Resonac Corporation, Tokyo (JP)

(21) Appl. No.: 18/860,947

(22) PCT Filed: Feb. 28, 2024
(86) PCT No.: PCT/JP2024/007360

§ 371 Date: Oct. 28, 2024

(30) Foreign Application Priority Data
Mar. 7, 2023 (JP) 2023-034983

(51) Int. Cl. G11B 5/725; C08G 65/333; C10M 107/44; C10N 40/18; C10N 50/08

(52) U.S. Cl. CPC G11B 5/7253; C08G 65/33396; C10M 107/44; C10M 2217/065; C10N 2040/18; C10N 2050/08

(57) ABSTRACT

Provided is a fluorine-containing ether compound represented by the following formula. R¹—O—R²—CH₂—R³—CH₂—R⁴—O—R⁵ (R³ represents a perfluoropolyether chain, R² and R⁴ represent a divalent linking group having one or more polar groups, R¹ and R⁵ represent Formula (2), Formula (3), or a hydrogen atom, and at least one of R¹ or R⁵ is represented by Formula (2) or (3), in Formula (2), X¹ represents an alkylene group having 1 to 30 carbon atoms, and Y and Z represent an aliphatic group having 1 to 30 carbon atoms, in Formula (3), X² represents an alkylene group having 1 to 30 carbon atoms, and A and B represent an aliphatic group having 1 to 30 carbon atoms.)

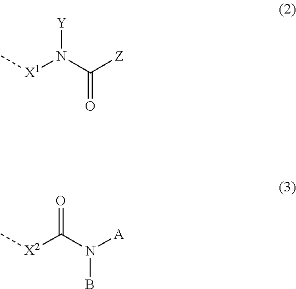

### TECHNICAL FIELD

The present invention relates to a fluorine-containing ether compound, a lubricant for a magnetic recording medium, and a magnetic recording medium.

Priority is claimed on Japanese Patent Application No. 2023-034983, filed Mar. 7, 2023, the content of which is incorporated herein by reference.

### BACKGROUND ART

In order to improve the recording density of a magnetic recording and reproducing device, development of a magnetic recording medium suitable for a high recording density has been promoted.

In the related art, as a magnetic recording medium, there is a magnetic recording medium in which a recording layer is formed on a substrate and a protective layer made of carbon or the like is formed on the recording layer. The protective layer protects information recorded on the recording layer and increases the slidability of a magnetic head. However, the durability of the magnetic recording medium is not sufficiently obtained only by providing the protective layer on the recording layer. Therefore, in general, a lubricating layer is formed by coating a surface of the protective layer with a lubricant.

As a lubricant used in a case of forming a lubricating layer of a magnetic recording medium, for example, a lubricant which contains a compound containing a polar group such as a hydroxy group or an amino group at an end of a fluorine-based polymer having a repeating structure containing —CF₂— has been proposed.

For example, as the lubricant, a perfluoropolyether compound having a plurality of hydroxy groups and a substituent at an end portion, in which the shortest distance between the hydroxy groups is separated by 3 or more atoms, is known (for example, see Patent Document 1).

In addition, a lubricant containing a fluoropolyether compound that contains an aromatic group and a hydroxy group is known (for example, see Patent Document 2). In addition, Patent Document 2 discloses a compound represented by Formula (1) in which an amide group is bonded to an end through a phenylene group.

In addition, Patent Document 3 discloses that, as the lubricant, a fluoropolyether compound which has a plurality of perfluoropolyether groups, an end group having at least one hydroxy group, and a linking group formed of a hydrocarbon group having at least one hydroxy group is used.

In addition, Patent Document 4 discloses a fluorine-containing ether compound in which divalent linking groups having a polar group are linked to both ends of a perfluoropolyether chain, and an end group in which one or more hydrogens of a chain organic group having 1 to 8 carbon atoms is substituted with a group having an amide bond is bonded to at least one of the divalent linking groups.

### CITATION LIST

### Patent Documents

Patent Document 1: Japanese Patent No. 4632144
Patent Document 2: Japanese Unexamined Patent Application, First Publication No. 2013-163667
Patent Document 3: Japanese Patent No. 6763980
Patent Document 4: PCT International Publication No. WO2019/039265

### SUMMARY OF INVENTION

### Technical Problem

In the magnetic recording and reproducing device, it is required to further reduce the floating amount of the magnetic head. Therefore, it is required to further reduce the thickness of the lubricating layer in the magnetic recording medium.

However, in general, in a case where the thickness of the lubricating layer is reduced, the chemical substance resistance and the wear resistance of the magnetic recording medium tend to be degraded. In addition, in a case where the thickness of the lubricating layer is reduced, the corrosion resistance of the magnetic recording medium may be insufficient. Therefore, there is a demand for a lubricating layer which has excellent chemical substance resistance, excellent wear resistance, and a high corrosion suppression effect of the magnetic recording medium.

The present invention has been made in consideration of the above-described circumstances, and an object thereof is to provide a fluorine-containing ether compound which can form a lubricating layer which has excellent chemical substance resistance, excellent wear resistance, and a high corrosion suppression effect of a magnetic recording medium and can be suitably used as a material for a lubricant for a magnetic recording medium.

Further, another object of the present invention is to provide a lubricant for a magnetic recording medium, which contains the fluorine-containing ether compound of the present invention and can form a lubricating layer which has satisfactory chemical substance resistance, satisfactory wear resistance, and excellent corrosion resistance.

Further, still another object of the present invention is to provide a magnetic recording medium which has a lubricating layer containing the fluorine-containing ether compound of the present invention and has satisfactory chemical substance resistance, satisfactory wear resistance, and excellent corrosion resistance.

### Solution to Problem

The present invention relates to the following aspects.

[1] A fluorine-containing ether compound represented by Formula (1).

R¹—O—R²—CH₂—R³—CH₂—R⁴—O—R⁵ (1)

(In Formula (1), R³ represents a perfluoropolyether chain. R² and R⁴ represent a divalent linking group having one or more polar groups, and may be the same as or different from each other. R¹ and R⁵ represent an end group represented by Formula (2), an end group represented by Formula (3), or a hydrogen atom, where R¹ and R⁵ may be the same as or different from each other. At least one of R¹ or R⁵ represents the end group represented by Formula (2) or (3).)

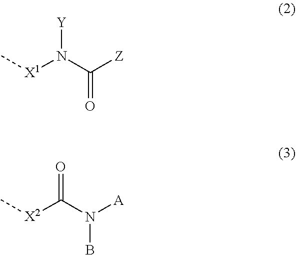

(In Formula (2), X¹ represents an alkylene group having 1 to 30 carbon atoms. Y and Z each independently represent an aliphatic group having 1 to 30 carbon atoms, which may have a polar group or an ether oxygen atom. Y and Z may be bonded to each other to form a cyclic structure.)

(In Formula (3), X² represents an alkylene group having 1 to 30 carbon atoms. A and B each independently represent an aliphatic group having 1 to 30 carbon atoms, which may have a polar group or an ether oxygen atom. A and B may be bonded to each other to form a cyclic structure.)

[2] The fluorine-containing ether compound according to [1], in which R¹ and R⁵ in Formula (1) each independently represent the end group represented by Formula (2) or (3).

[3] The fluorine-containing ether compound according to [2], in which R¹ and R⁵ in Formula (1) are the same as each other.

[4] The fluorine-containing ether compound according to [2], in which R¹ and R⁵ in Formula (1) are different from each other.

[5] The fluorine-containing ether compound according to [1], in which one of R¹ or R⁵ in Formula (1) represents the end group represented by Formula (2) or (3), and the other represents a hydrogen atom.

[6] The fluorine-containing ether compound according to any one of [1] to [5], in which R²—O— and —R⁴—O— in Formula (1) are each independently represented by Formula (4).

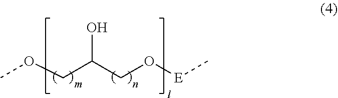

(In Formula (4), 1 represents an integer of 1 to 3. 1 pieces of m's each independently represent an integer of 1 to 6. 1 pieces of n's each independently represent an integer of 1 to 6. In one repeating unit, at least one of m or n represents 1. E represents a single bond, —CH₂CH₂O— (a leftmost carbon atom is bonded to an oxygen atom in a repeating unit), —CH₂CH₂CH₂O— (a leftmost carbon atom is bonded to an oxygen atom in a repeating unit), or —CH₂CH₂CH₂CH₂O— (a leftmost carbon atom is bonded to an oxygen atom in a repeating unit). In Formula (4), a leftmost oxygen atom is bonded to a methylene group bonded to R³, and E is bonded to R¹ or R⁵.)

[7] The fluorine-containing ether compound according to any one of [1] to [6], in which —R²—O— and —R⁴—O— in Formula (1) are each independently represented by Formula (5-1) or (5-2).

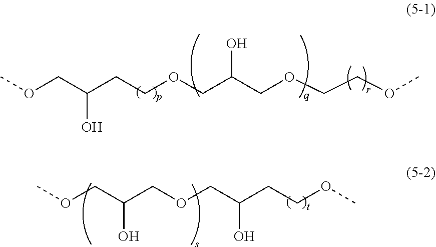

(In Formula (5-1), p represents an integer of 0 to 3, q represents an integer of 0 to 2, and r represents an integer of 1 to 3. In Formula (5-1), a leftmost oxygen atom is bonded to a methylene group bonded to R³, and a rightmost oxygen atom is bonded to R¹ or R⁵.)

(In Formula (5-2), s represents an integer of 0 to 2, and t represents an integer of 0 to 3. In Formula (5-2), a leftmost oxygen atom is bonded to the methylene group bonded to R³, and a rightmost oxygen atom is bonded to R¹ or R⁵.)

[8] The fluorine-containing ether compound according to any one of [1] to [7], in which R³ in Formula (1) represents a perfluoropolyether chain represented by Formula (6).

—(CF₂)_{w1}—O—(CF₂O)_{w2}—(CF₂CF₂O)_{w3}—(CF₂CF₂CF₂O)_{w4}—(CF₂CF₂CF₂CF₂O)_{w5}—(CF₂)_{w6}— (6)

(In Formula (6), w2, w3, w4, and w5 represent an average degree of polymerization, and each independently represent 0 to 20. Here, all of w2, w3, w4, and w5 do not represent 0 at the same time. w1 and w6 represent an average value representing the number of CF₂'s, and each independently represent 1 to 3. An arrangement order of (CF₂O), (CF₂CF₂O), (CF₂CF₂CF₂O), and (CF₂CF₂CF₂CF₂O), which are repeating units in Formula (6), is not particularly limited.)

[9] The fluorine-containing ether compound according to any one of [1] to [8], in which R³ in Formula (1) represents any one selected from the group consisting of perfluoropolyether chains represented by any one of Formulae (7-1) to (7-4).

—CF₂—(OCF₂CF₂)ₕ—(OCF₂)ᵢ—OCF₂— (7-1)

(In Formula (7-1), h and i represent an average degree of polymerization, where h represents 1 to 20 and i represents 0 to 20.)

—CF₂CF₂—(OCF₂CF₂CF₂)ₖ—OCF₂CF₂— (7-2)

(In Formula (7-2), j represents an average degree of polymerization, and represents 1 to 15.)

—CF₂CF₂CF₂—(OCF₂CF₂CF₂CF₂)ₖ—OCF₂CF₂CF₂— (7-3)

(In Formula (7-3), k represents an average degree of polymerization, and represents 1 to 10.)

—(CF₂)_{w7}—O—(CF₂CF₂CF₂O)_{w8}—(CF₂CF₂O)_{w9}—(CF₂)_{w10}— (7-4)

(In Formula (7-4), w8 and w9 represent an average degree of polymerization, and each independently represents 1 to 20. w7 and w10 represent an average value representing the number of CF₂'s, and each independently represent 1 to 2.)

[10] The fluorine-containing ether compound according to any one of [1] to [9], in which the fluorine-containing ether compound has a number-average molecular weight of 500 to 10000.

[11] A lubricant for a magnetic recording medium, including: the fluorine-containing ether compound according to any one of [1] to [10].

[12] A magnetic recording medium, which is provided with at least a magnetic layer, a protective layer, and a lubricating layer in this order on a substrate,
in which the lubricating layer contains the fluorine-containing ether compound according to any one of [1] to [10].

[13] The magnetic recording medium according to [12], in which the lubricating layer has an average film thickness of 0.5 nm to 2.0 nm.

### Advantageous Effects of Invention

The fluorine-containing ether compound of the present invention is a compound represented by Formula (1), and is suitable as a material of a lubricant for a magnetic recording medium.

Since the lubricant for a magnetic recording medium of the present invention contains the fluorine-containing ether compound of the present invention, a lubricating layer having satisfactory chemical substance resistance, satisfactory wear resistance, and a high corrosion suppression effect of the magnetic recording medium can be formed.

The magnetic recording medium of the present invention includes a lubricating layer containing the fluorine-containing ether compound of the present invention. Therefore, the magnetic recording medium of the present invention has satisfactory chemical substance resistance and wear resistance, excellent corrosion resistance, and excellent reliability and durability. In addition, since the lubricating layer of the magnetic recording medium of the present invention has satisfactory chemical substance resistance and wear resistance and a high corrosion suppression effect of the magnetic recording medium, the thickness of the lubricating layer can be reduced.

### BRIEF DESCRIPTION OF DRAWINGS

FIG. 1A schematic cross-sectional view showing an embodiment of a magnetic recording medium according to the present invention.

### DESCRIPTION OF EMBODIMENTS

In order to solve the above-described problems, the present inventors have conducted intensive examination as described below.

In the related art, as a material of a lubricant for a magnetic recording medium (hereinafter, also referred to as “lubricant”) which is applied to a surface of a protective layer, a fluorine-containing ether compound that contains a polar group such as a hydroxy group at an end of a chain structure has been preferably used. The polar group in the fluorine-containing ether compound is bonded to the active point on the protective layer to improve adhesion of the lubricating layer to the protective layer. Therefore, as the material of the lubricant, a fluorine-containing ether compound containing a polar group in the chain structure in addition to an end of the chain structure is particularly preferably used.

However, in a case where a lubricating layer having a small thickness is formed on the protective layer using a lubricant of the related art, as described below, it is difficult to realize a lubricating layer having satisfactory chemical substance resistance and wear resistance and a high corrosion suppression effect of the magnetic recording medium.

That is, in a case where the adhesion of the lubricant to the protective layer is insufficient, a state of the lubricant applied onto the protective layer is bulky. Therefore, a state of coating the protective layer with the lubricating layer is likely to be uneven. In a case where the state of coating with the lubricating layer is uneven, the chemical substance resistance and the corrosion resistance of the lubricating layer are insufficient. Therefore, in a case where the adhesion of the lubricant to the protective layer is insufficient, sufficient chemical substance resistance and corrosion resistance cannot be obtained unless the film thickness is increased and the state of coating the protective layer with the lubricating layer is made uniform.

In addition, in a case where the adhesion of the lubricant to the protective layer is weak, a polar group in the fluorine-containing ether compound which is not involved in the bonding to the active point on the protective layer is generated, and water, which causes environmental substances generating contaminants and corrosion of the magnetic recording medium, is attracted to the lubricating layer. As a result, the chemical substance resistance of the lubricating layer and the corrosion resistance of the magnetic recording medium are deteriorated.

As a method for improving the adhesion of the lubricant to the protective layer, it is considered that a fluorine-containing ether compound having a plurality of polar groups in which the polar groups are bonded to carbon atoms at both most ends of a chain structure and other carbon atoms in the chain structure is used as a material of the lubricant.

However, in the lubricating layer formed of such a fluorine-containing ether compound, the adhesion to the protective layer is extremely strong so that the fluidity is insufficient and the lubricity is impaired, and thus the wear resistance may be insufficient.

In addition, in the lubricating layer formed of such a fluorine-containing ether compound, since the hydrophilicity of the lubricant is extremely high, water easily penetrates, and corrosion of the magnetic recording medium may be observed.

Therefore, the present inventors have focused on a bond between a polar group contained in the fluorine-containing ether compound and an active point on the protective layer. Further, the present inventors have repeatedly conducted intensive examination in order to achieve a fluorine-containing ether compound which has a uniform state of coating the protective layer, is unlikely to generate a polar group which is not involved in the bonding to the active point on the protective layer, and has moderate and satisfactory adhesion, to realize a fluorine-containing ether compound capable of forming a lubricating layer having satisfactory chemical substance resistance and wear resistance and a high corrosion suppression effect of a magnetic recording medium.

As a result, it has been found that a fluorine-containing ether compound, in which a methylene group (—CH₂—), a divalent linking group having one or more polar groups, an oxygen atom, and an end group having an N,N-substituted amide (hereinafter, also simply referred to as “N,N-substituted amide”) in which a specific aliphatic group is bonded to a nitrogen atom constituting an amide bond and a hydrogen atom is not bonded to the nitrogen atom, and a hydrogen atom are bonded in this order to both sides of a perfluoropolyether chain, and the end group having the N,N-substituted amide is disposed at least one end, may be obtained.

In such a fluorine-containing ether compound, it is presumed that a lubricating layer having moderately satisfying adhesion in which a polar group which is not involved in the bonding to the active point on the protective layer is unlikely to be generated, can be formed by <1> described below, a lubricating layer having a uniform state of coating the protective layer can be formed by <2> described below, and a lubricating layer having satisfactory fat-solubility can be formed by <3> described below, and thus a lubricating layer having satisfactory chemical substance resistance and wear resistance and a high corrosion suppression effect of the magnetic recording medium can be obtained.

<1> The fluorine-containing ether compound has an end group having a specific N,N-substituted amide bonded to at least one end. A bond between a carbonyl carbon atom constituting the amide bond of the N,N-substituted amide of the end group and a carbon atom adjacent to the nitrogen atom is difficult to freely rotate. Therefore, the N,N-substituted amide of the end group disposed at least one end and the polar group of the divalent linking group adjacent to the end group are unlikely to interact with each other.

In addition, in the fluorine-containing ether compound, the N,N-substituted amide of the end group disposed at least one end is a structure in which specific aliphatic groups are bonded to a nitrogen atom constituting an amide bond and a hydrogen atom is not bonded to the nitrogen atom. Therefore, in the above-described fluorine-containing ether compound, for example, the interaction between the N,N-substituted amide of the end group disposed at least one end and the polar group of the divalent linking group adjacent to the end group is more difficult to form as compared with a case where one or two hydrogen atoms are bonded to a nitrogen atom constituting an amide bond.

In addition, in the fluorine-containing ether compound, the N,N-substituted amide of the end group disposed at least one end and the polar group of the divalent linking group adjacent to the end group have an extremely low ability to inhibit the interaction with the protective layer. Therefore, in the above-described fluorine-containing ether compound, the N,N-substituted amide of the end group disposed at least one end and the polar group of the divalent linking group adjacent to the end group are each likely to be involved in the bonding with the active point on the protective layer.

In addition, the N,N′-substituted amide of the end group disposed at least one end exhibits a moderate interaction with the protective layer, and the polar groups of the divalent linking group adjacent to the end group having the N,N′-substituted amide each independently exhibit a satisfactory interaction with the protective layer. As a result, the N,N-substituted amide of the end group disposed at least one end and the polar group of the divalent linking group adjacent to the N,N-substituted amide of the end group can each independently be bonded to a plurality of functional groups (active points) on the protective layer. Therefore, a polar group which is not bonded to the active point on the protective layer is unlikely to be generated, and the number of polar groups which are not involved in the bonding with the active point on the protective layer is suppressed. Therefore, the fluorine-containing ether compound can form a lubricating layer having moderately satisfactory adhesion to the protective layer and suppresses a polar group which is not involved in the bonding to the active point on the protective layer in the compound from attracting an environmental substance generating a contaminant, and water, which causes corrosion of the magnetic recording medium, to the lubricating layer.

<2> The fluorine-containing ether compound has a structure in which specific aliphatic groups are bonded to a nitrogen atom constituting an amide bond of N,N-substituted amide of an end group disposed at least one end, and a hydrogen atom is not bonded to the nitrogen atom. Therefore, the above-described fluorine-containing ether compound is, for example, unlikely to be aggregated as compared with a case where one or two hydrogen atoms are bonded to a nitrogen atom constituting an amide bond. This is because a polar group of the divalent linking group contained in the above-described fluorine-containing ether compound and the N,N-substituted amide are unlikely to interact with each other (in a case where end groups having N,N-substituted amide are disposed at both ends, the polar group of the divalent linking group, the N,N-substituted amide, and the N,N-substituted amides are unlikely to interact with each other).

In addition, the N,N-substituted amide of the end group disposed at least one end is unlikely to inhibit adhesion of the polar group having a divalent linking group to the protective layer. In addition, in a case where the end groups having N,N′-substituted amide are disposed at both ends, the N,N′-substituted amides are unlikely to inhibit adhesion to the protective layer.

Therefore, the above-described fluorine-containing ether compound easily wets and spreads on the protective layer, and a lubricating layer having a uniform film state can be formed.

<3> In the end group having an N,N-substituted amide, which is contained in the fluorine-containing ether compound, specific aliphatic groups are bonded a nitrogen atom constituting an amide bond and a hydrogen atom is not bonded to the nitrogen atom. Therefore, the above-described fluorine-containing ether compound has satisfactory fat-solubility as compared with, for example, a case where hydrogen atoms in place of end groups having a N,N-substituted amide are disposed (in other words, both ends are hydrogen atoms) and a case where one or two hydrogen atoms are bonded to a nitrogen atom constituting an amide bond. Therefore, the above-described fluorine-containing ether compound can effectively suppress penetration of water, which causes corrosion of the magnetic recording medium, and can form a lubricating layer having a high corrosion suppression effect of the magnetic recording medium.

Further, the present inventors have confirmed that, by forming a lubricating layer on a protective layer of a magnetic recording medium using the above-described lubricant containing the fluorine-containing ether compound, a lubricating layer having satisfactory chemical substance resistance and wear resistance and excellent corrosion resistance can be formed, thereby completing the present invention.

Hereinafter, the fluorine-containing ether compound, the lubricant for a magnetic recording medium, and the magnetic recording medium of the present invention will be described in detail. Further, the present invention is not limited to the embodiments described below. In the present invention, the number, the amount, the ratio, the composition, the kind, the position, the material, the configuration, and the like can be added, omitted, substituted, or changed within a range where the gist of the present invention is not departed.

### [Fluorine-Containing Ether Compound]

There is provided a fluorine-containing ether compound represented by Formula (1).

R¹—O—R²—CH₂—R³—CH₂—R⁴—O—R⁵ (1)

(In Formula (1), R³ represents a perfluoropolyether chain. R² and R⁴ represent a divalent linking group having one or more polar groups, and may be the same as or different from each other. R¹ and R⁵ represent an end group represented by Formula (2), an end group represented by Formula (3), or a hydrogen atom, where R¹ and R⁵ may be the same as or different from each other. At least one of R¹ or R⁵ represents an end group represented by Formula (2) or (3)).

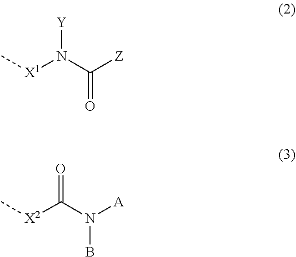

(In Formula (2), X¹ represents an alkylene group having 1 to 30 carbon atoms. Y and Z each independently represent an aliphatic group having 1 to 30 carbon atoms, which may have a polar group or an ether oxygen atom. Y and Z may be bonded to each other to form a cyclic structure.)

(In Formula (3), X² represents an alkylene group having 1 to 30 carbon atoms. A and B each independently represent an aliphatic group having 1 to 30 carbon atoms, which may have a polar group or an ether oxygen atom. A and B may be bonded to each other to form a cyclic structure.)

(Divalent Linking Group Represented by R² and R⁴)

In the fluorine-containing ether compound represented by Formula (1), R² and R⁴ each represent a divalent linking group having one or more polar groups. R² and R⁴ may be the same as or different from each other.

Examples of the polar group included in R² and R⁴ include a hydroxy group (—OH), a cyano group (—CN), an amino group (—NH₂), a carboxy group (—COOH), a formyl group (—((C═O)H), a carbonyl group (—CO—), and a sulfo group (—SO₃H). Among these, as the polar group, a hydroxy group is preferable. The hydroxy group has a large interaction with a protective layer, particularly a protective layer formed of a carbon-based material. Therefore, in a case where the polar group included in R² and R⁴ is a hydroxy group, the lubricating layer containing the fluorine-containing ether compound has higher adhesion to the protective layer.

The numbers of polar groups included in R² and R⁴ are each preferably 1 to 3. Since the number of polar groups is 1 or more, in a case where a lubricating layer is formed on the protective layer using the lubricant containing the fluorine-containing ether compound according to the present embodiment, suitable interaction occurs between the lubricating layer and the protective layer. In a case where the number of polar groups included in R² and R⁴ is 3 or less, the number of polar groups included in R² and R⁴ is large, so that the hydrophilicity of the fluorine-containing ether compound is increased, and the fluorine-containing ether compound allows the penetration of water, which causes corrosion of the magnetic recording medium, in the lubricating layer containing the fluorine-containing ether compound, which can be suppressed.

In addition, since the number of polar groups included in the fluorine-containing ether compound represented by Formula (1) is likely to be 4 to 6, the numbers of polar groups included in R² and R⁴ are each preferably 1 or 2.

R² and R⁴ each represent preferably a linking group having 3 to 15 carbon atoms and more preferably represent a linking group having 3 to 10 carbon atoms. In a case where R² and R⁴ represent a linking group having 3 or more carbon atoms, the compound is likely to be a fluorine-containing ether compound having sufficient hydrophobicity, and water, which causes corrosion of the magnetic recording medium, can be effectively suppressed from penetrating, and thus a lubricating layer having a high corrosion suppression effect can be formed. In addition, in a case where R² and R⁴ represent a linking group having 15 or less carbon atoms, the hydrophobicity of the fluorine-containing ether compound represented by Formula (1) is too high, and the interaction between the lubricating layer and the protective layer is weakened, and the adhesion of the lubricating layer to the protective layer is lowered, which can be suppressed.

R² is bonded to R¹ through an oxygen atom. Therefore, in a case where R¹ is represented by Formula (2) or (3), R² and R¹ are bonded to each other by an ether bond. In a case where R¹ represents a hydrogen atom, an oxygen atom bonded to R² and R¹ form a hydroxy group. It is preferable that an end of R² on the side bonded to the methylene group adjacent to R³ is an oxygen atom.

R⁴ is bonded to R⁵ through an oxygen atom. Therefore, in a case where R⁵ is represented by Formula (2) or (3), R⁴ and R⁵ are bonded to each other by an ether bond. In a case where R⁵ represents a hydrogen atom, the oxygen atom bonded to R⁴ and R⁵ form a hydroxy group. It is preferable that an end of R⁴, which is bonded to the methylene group adjacent to R³, is an oxygen atom.

It is preferable that —R²—O— and —R⁴—O— in Formula (1) are each independently represented by Formula (4).

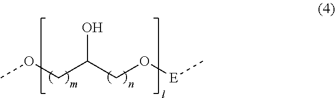

(In Formula (4), 1 represents an integer of 1 to 3. 1 pieces of m's each independently represent an integer of 1 to 6. 1 pieces of n's each independently represent an integer of 1 to 6. In one repeating unit, at least one of m or n represents 1. E represents a single bond, —CH₂CH₂O— (a leftmost carbon atom is bonded to an oxygen atom in a repeating unit), —CH₂CH₂CH₂O— (a leftmost carbon atom is bonded to an oxygen atom in a repeating unit), or —CH₂CH₂CH₂CH₂O— (a leftmost carbon atom is bonded to an oxygen atom in a repeating unit). In Formula (4), a leftmost oxygen atom is bonded to a methylene group bonded to R³, and E is bonded to R¹ or R⁵.)

1 in Formula (4) is an integer of 1 to 3. Therefore, Formula (4) has 1 to 3 repeating units (—((CH₂)ₘ—CH(OH)—(CH₂)ₙ—O—). Each repeating unit in Formula (4) has one secondary hydroxy group which interacts with the protective layer. Moreover, each repeating unit in Formula (4) has an oxygen atom at an end on the side bonded to R or R⁵. In addition, in Formula (4), an end on the side bonded to the methylene group bonded to R³ is bonded to the methylene group bonded to R³ by an ether bond. Therefore, Formula (4) has moderate flexibility. From these facts, the one to three secondary hydroxy groups included in Formula (4) each likely to independently be involved in the bonding with the large number of active points present on the protective layer, and thus the secondary hydroxy groups have excellent adhesion to the protective layer.

Since 1 in Formula (4) is 3 or less, in a case where the number of hydroxy groups in Formula (4) is too large, water, which causes corrosion, is attracted to the lubricating layer containing the fluorine-containing ether compound, and thus it is possible to prevent the attraction of water and to form a lubricating layer having a high effect of suppressing corrosion of the magnetic recording medium. Since a lubricating layer having a more excellent corrosion suppressing effect can be obtained, I in Formula (4) is preferably an integer of 1 or 2, and most preferably 1.

In a case where 1 in Formula (4) is 2 or 3, combinations of m's and n's in two or three repeating units (—((CH₂)ₘ—CH(OH)—(CH₂)ₙ—O—) may be partially or entirely the same as or different from each other.

In a case where 1 in Formula (4) is 2 or 3, two or three hydroxy groups in Formula (4) are bonded to different carbon atoms, respectively. In addition, the carbon atoms to which the hydroxy groups are bonded are bonded to each other through a linking chain including a carbon atom to which the hydroxy group is not bonded. Therefore, the two or three hydroxy groups in Formula (4) can be aligned in such a manner that all of the hydroxy groups in Formula (4) are aligned by a linking chain including an unbound carbon atom of the hydroxy group in Formula (4), and can be aligned to be in close contact with the protective layer. Therefore, all of the two or three hydroxy groups in Formula (4) are likely to be involved in a bond with a large number of active points present on the protective layer.

In addition, in a case where 1 in Formula (4) is 2 or 3, the carbon atom included in the linking chain arranged between the hydroxy groups bonded to each other prevents the intramolecular interaction between the adjacent hydroxy groups from occurring preferentially to the interaction between the hydroxy group and the protective layer, and thus improves the adhesion between the hydroxy group in Formula (4) and the protective layer.

In addition, in a case where 1 in Formula (4) is 2 or 3, a linking chain between the hydroxy groups bonded to the carbon atoms has a linear structure consisting of 3 or more atoms including at least two carbon atoms not bonded to a hydroxy group. Therefore, even in a case where the linking chain between the carbon atoms to which the hydroxy groups are bonded contains an oxygen atom forming an ether bond, the fluorine-containing ether compound has satisfactory hydrophobicity. In addition, since the above-described linking chain has a linear structure composed of three or more atoms, the molecular mobility is appropriate, the intramolecular aggregation is unlikely to occur, and the protective layer has excellent adhesion.

1 pieces of m's in Formula (4) each independently represent an integer of 1 to 6, and 1 pieces of n's each independently represent an integer of 1 to 6. Since 1 pieces of m's and 1 pieces of n's in Formula (4) each represent 1 or more, the fluorine-containing ether compound has sufficient hydrophobicity, and thus water, which causes corrosion of the magnetic recording medium, can be effectively suppressed from penetrating, and a lubricating layer having a high corrosion suppression effect can be formed.

In addition, in one repeating unit of Formula (4), at least one of m or n is 1. This is because, in a case where the number of carbon atoms in the alkylene group between the carbon atom bonded to the hydroxy group and the ether oxygen atom is too large, the mobility of the hydroxy group included in Formula (4) is not reduced, and the interaction with the protective layer is likely to occur.

In one repeating unit of Formula (4), since the moiety having a value of m or n which is not 1 is 6 or less, the rigid alkylene chain between the carbon atom to which the hydroxy group is bonded and the ether oxygen atom is long, the flexibility of Formula (4) is lowered, and the interaction with the protective layer is weakened, so that the portion is prevented from floating.

In one repeating unit of Formula (4), the number of the m or n which is not 1 is preferably 4 or less. The reason for this is the hydrophobicity of Formula (4) is extremely high, and thus hindrance of the adhesion to the protective layer can be prevented. In addition, this is because the hydroxy group represented by formula (4) is not extremely bulky, and thus hindrance of movement of the hydroxy group in the fluorine-containing ether compound can be suppressed from increasing. In addition, in a case where the number of m or n, which is not 1, is 4 or less, the rigid alkylene chain in the main chain portion of Formula (4) is too long, so that the flexibility of Formula (4) is lowered and the interaction with the protective layer is lowered. From these facts, in a case where the number of m and n which are not 1 is 4 or less, the hydroxy groups in the fluorine-containing ether compound is are likely to be independently involved in bonding to the active point on the protective layer.

In one repeating unit of Formula (4), the number of m and n that are not 1 is preferably 3 or less, more preferably 2 or less, and most preferably both m and n are 1.

In one repeating unit of Formula (4), the number of m or n which is not 1 can be appropriately selected according to the type of R¹ (R⁵) in Formula (1), the performance required for the lubricant containing the fluorine-containing ether compound, and the like.

For example, in a case where n of the repeating unit in Formula (4) which is arranged on the most molecular end side (side bonded to R¹ or R⁵) is 2 or more, the distance between the hydroxy group in Formula (4) and R¹ (R⁵) in Formula (1) may be more appropriate in a case where E is a single bond. Therefore, in a case where E is a single bond, n of the repeating unit represented by Formula (4) which is arranged on the most molecular end side may be preferably 2 to 4 and more preferably 2 or 3.

E in Formula (4) is any of a single bond, —CH₂CH₂O—, —CH₂CH₂CH₂O—, or —CH₂CH₂CH₂CH₂O—, and can be appropriately selected according to the type of R¹ (R⁵) in Formula (1), the performance required for the lubricant containing the fluorine-containing ether compound, and the like.

In a case where E is a single bond, in Formula (4), the oxygen atom on the most molecular end side of the repeating unit (—(CH₂)ₘ—CH(OH)—(CH₂)ₙ—O—) (side bonded to R¹ or R⁵) is directly bonded to R¹ or R⁵.

In a case where E in Formula (4) is any of —CH₂CH₂O—, —CH₂CH₂CH₂O—, or —CH₂CH₂CH₂CH₂O—, the fluorine-containing ether compound represented by Formula (1) has higher hydrophobicity, and thus a lubricating layer having a higher corrosion suppressing effect can be formed.

In addition, in a case where E in Formula (4) is any of —CH₂CH₂O—, —CH₂CH₂CH₂O—, or —CH₂CH₂CH₂CH₂O—, a distance between the hydroxy group in Formula (4) and R¹ (R⁵) in Formula (1) may be more appropriate. As a result, interaction between hydroxy groups in the fluorine-containing ether compound molecule can be suppressed, and the hydroxy groups in the fluorine-containing ether compound represented by Formula (1) may be easily adhered to the protective layer.

E in Formula (4) is preferably a single bond, —CH₂CH₂O—, or —CH₂CH₂CH₂O—, and more preferably a single bond or —CH₂CH₂O—. This is because, in a case where the hydrophobicity of the fluorine-containing ether compound represented by Formula (1) is too high, the interaction between the lubricating layer and the protective layer is weakened, and the adhesion of the lubricating layer to the protective layer is lowered.

It is preferable that 1 in Formula (4), n of the repeating unit which is on the most molecular end side in Formula (4), and E are determined according to the type of R¹ (R⁵) in Formula (1).

Specifically, in a case where R¹ (R⁵) in Formula (1) is the end group represented by Formula (2) or the end group represented by Formula (3) and —R²—O—(—R⁴—O—) is Formula (4), 1 in Formula (4) is preferably 1 or 2. In this case, the number of secondary hydroxy groups in Formula (4) is 1 or 2. Therefore, even in a case where R¹ (R⁵) is the end group represented by Formula (2) or the end group represented by Formula (3), the hydrophobicity of the fluorine-containing ether compound represented by Formula (1) is too high, and the interaction between the lubricating layer and the protective layer is weakened, and the adhesion of the lubricating layer to the protective layer is lowered, which may lead to a decrease in wear resistance, and the abrasion resistance can be suppressed.

In addition, in a case where R¹ (R⁵) in Formula (1) is a hydrogen atom and —R²—O—(—R⁴—O—) is Formula (4), 1 in Formula (4) is preferably 1 or 2, and is most preferably 1. This is because, even in a case where the oxygen atom of Formula (4) which is disposed on the most molecular end side and R¹ (R⁵) which is a hydrogen atom form a hydroxy group, the number of hydroxy groups in the fluorine-containing ether compound represented by Formula (1) is too large, the hydrophilicity is increased, and water which causes corrosion is prevented from being attracted to the lubricating layer, so that a lubricating layer having excellent corrosion resistance is obtained.

In addition, in a case where R¹ (R⁵) in Formula (1) is the end group represented by Formula (2) or the end group represented by Formula (3), and —R²—O—(—R⁴—O—) is Formula (4), E may be any of a single bond, —CH₂CH₂O—, —CH₂CH₂CH₂O—, or —CH₂CH₂CH₂CH₂O—, and 1 pieces of n's each may be any of integers of 1 to 6. This is because, even in a case where E is a single bond and n of the repeating unit in Formula (4) which is disposed on the most molecular end side is 1, the alkylene group represented by X¹ in Formula (2) or X² in Formula (3) is arranged between the hydroxy group arranged on the most molecular end side in Formula (4) and the N,N-substituted amide.

Therefore, the hydroxy group in Formula (4) is unlikely to aggregate with N,N-substituted amide in a case where R¹ (R⁵) is an end group represented by Formula (2) or Formula (3). Therefore, the hydroxy group in Formula (4) is likely to interact with the protective layer, regardless of whether R¹ (R⁵) is the end group represented by Formula (2) or the end group represented by Formula (3). From this, in a case where R¹ (R⁵) is the end group represented by Formula (2) or Formula (3) and —R²—O—(—R⁴—O—) is Formula (4), for example, in a case where the carbon atom to which the hydroxy group in the divalent linking group represented by R² (R⁴) is bonded (carbon atom in —CH(OH)—) and R¹ (R⁵) are bonded only through an oxygen atom, the partial structure of —R²—O—(—R⁴—O—) included in the fluorine-containing ether compound is unlikely to be exposed, and a lubricating layer having satisfactory adhesion to the protective layer can be formed.

On the other hand, in a case where R¹ (R⁵) in Formula (1) is a hydrogen atom and —R²—O—(—R⁴—O—) is Formula (4), it is preferable that E is —CH₂CH₂O—, —CH₂CH₂CH₂O—, or —CH₂CH₂CH₂CH₂O—, and/or n of the repeating unit in Formula (4) which is disposed on the most molecular end side is 2 to 4. This is because an alkylene chain having 2 or more carbon atoms is arranged between the hydroxy group arranged on the most molecular end side in Formula (4) and R¹ (R⁵) in Formula (1).

As a result, the hydroxy group formed of the oxygen atom and R¹ (R⁵) as the hydrogen atom which are arranged on the most molecular end side in Formula (4) is unlikely to aggregate with the hydroxy group disposed on the most molecular end side in Formula (4). In addition, hydrophobicity is improved by the alkylene chain included in E in Formula (4) and/or the alkylene chain included in the repeating unit arranged on the most molecular end side in Formula (4). Therefore, even in a case where the oxygen atom of Formula (4) which is disposed on the most molecular end side and R¹ (R⁵) which is a hydrogen atom are to form a hydroxy group, the number of hydroxy groups in the fluorine-containing ether compound represented by Formula (1) is too large, so that the hydrophilicity is high, and water which causes corrosion is prevented from being attracted to the lubricating layer, and thus a lubricating layer having excellent corrosion resistance is obtained.

—R²—O— and —R⁴—O— in Formula (1) each independently are more preferably represented by Formula (5-1) or (5-2).

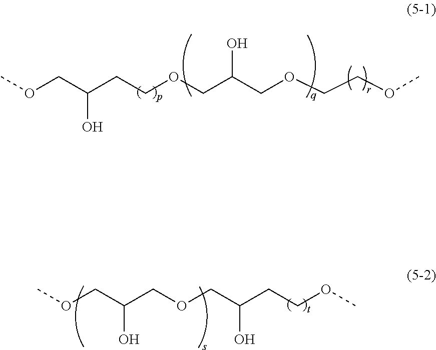

(In Formula (5-1), p represents an integer of 0 to 3, q represents an integer of 0 to 2, and r represents an integer of 1 to 3. In Formula (5-1), a leftmost oxygen atom is bonded to a methylene group bonded to R³, and a rightmost oxygen atom is bonded to R¹ or R⁵.)

(In Formula (5-2), s represents an integer of 0 to 2, and t represents an integer of 0 to 3. In Formula (5-2), a leftmost oxygen atom is bonded to the methylene group bonded to R³, and a rightmost oxygen atom is bonded to R¹ or R⁵.)

In Formula (5-1), 1 in Formula (4) is an integer of 1 to 3, m is 1, n of the repeating unit in Formula (4) which is most on the R³ side is 1 to 4, n of the repeating unit in Formula (4) which is most on the molecular end side is 1, and E is any of —CH₂CH₂O—, —CH₂CH₂CH₂O—, or —CH₂CH₂CH₂CH₂O—.

In Formula (5-2), 1 in Formula (4) is an integer of 1 to 3, m is 1, n of the repeating unit in Formula (4) which is on the most R³ side is 1, n of the repeating unit in Formula (4) which is on the most molecular end side is 1 to 4, and E is a single bond.

q in Formula (5-1) and s in Formula (5-2) represent an integer of 0 to 2, and correspond to a number of repeating units in Formula (4) minus 1.

In a case where q in Formula (5-1) represents 1 or 2, or s in Formula (5-2) represents 1 or 2, two or three hydroxy groups in Formula (5-1) or (5-2) are all likely to be involved in bonding with the active points present in large numbers on the protective layer similarly to two or three hydroxy groups in Formula (4), and strong interaction with the protective layer is obtained, so that the protective layer is excellent in adhesion, and the molecular internal aggregation is unlikely to occur.

p in Formula (5-1) represents an integer of 0 to 3, and corresponds to a number of n−1 of the repeating unit in Formula (4) which is disposed closest to R³ in Formula (4). p represents preferably 0 or 1 and more preferably 0.

In Formula (5-1), since the total value of p and r is 6 or less, the alkylene chain of the main chain portion of Formula (5-1) is not too long. Therefore, in a case where the rigid alkylene chain is long, the flexibility of the portion represented by Formula (5-1) is reduced, and the interaction with the protective layer is weakened, so that the floating of the portion represented by Formula (5-1) can be prevented. r represents preferably 1 or 2 and more preferably 1.

t in Formula (5-2) represents an integer of 0 to 3, and corresponds to a number of (n−1) which is one less than n of the repeating unit arranged on the most molecular end side in Formula (4). t represents preferably 1 or 2 and more preferably 1.

(End group represented by R¹ and R⁵)

In the fluorine-containing ether compound represented by Formula (1), R¹ and R⁵ represent an end group represented by Formula (2), an end group represented by Formula (3), or a hydrogen atom.

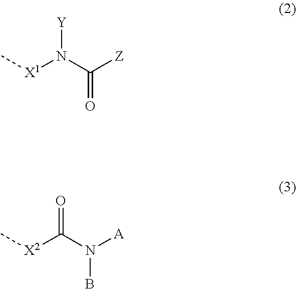

(In Formula (2), X¹ represents an alkylene group having 1 to 30 carbon atoms. Y and Z each independently represent an aliphatic group having 1 to 30 carbon atoms, which may have a polar group or an ether oxygen atom. Y and Z may be bonded to each other to form a cyclic structure.)

(In Formula (3), X² represents an alkylene group having 1 to 30 carbon atoms. A and B each independently represent an aliphatic group having 1 to 30 carbon atoms, which may have a polar group or an ether oxygen atom. A and B may be bonded to each other to form a cyclic structure.)

In the fluorine-containing ether compound represented by Formula (1), at least one of R¹ or R⁵ is an end group represented by Formula (2) or (3).

In a case where both of R¹ and R⁵ are the end groups represented by Formula (2) or Formula (3), the fluorine-containing ether compound in which the effect due to the N, N-substituted amide of Formula (2) and Formula (3) is more remarkable is obtained, and a lubricating layer having more excellent wear resistance and corrosion resistance is easily obtained.

In the fluorine-containing ether compound represented by Formula (1), in a case where both R¹ and R⁵ are the end groups represented by Formula (2) or (3), R¹ and R⁵ may be the same as or different from each other. In a case where R¹ and R⁵ are the same, a coating state of the protective layer with respect to the lubricating layer containing the fluorine-containing ether compound is more uniform, and a lubricating layer having better adhesion can be formed.

In the fluorine-containing ether compound represented by Formula (1), in a case where only one of R¹ or R⁵ (for example, R¹) is the end group represented by Formula (2) or (3), the other (for example, R⁵) is a hydrogen atom. In this case, a hydrogen atom disposed in R¹ or R⁵ which is not the end group represented by Formula (2) or (3) forms an oxygen atom and a hydroxy group by bonding to R² or R⁴. Therefore, in this fluorine-containing ether compound, a hydroxy group having a large interaction with the protective layer is disposed at one end, and thus a lubricating layer having excellent adhesion to the protective layer can be formed.

In the fluorine-containing ether compound represented by Formula (1), a bond of a carbonyl carbon atom constituting an amide bond of the N,N-substituted amide of Formula (2) and Formula (3) or a carbon atom adjacent to the nitrogen atom is difficult to freely rotate. Therefore, the N,N′-substituted amide represented by Formulae (2) and (3) and the polar group included in the divalent linking group represented by R² and/or R⁴ adjacent to the N,N′-substituted amide are unlikely to interact with each other, and the N,N′-substituted amide is unlikely to inhibit adhesion of the protective layer of the polar group included in the divalent linking group represented by R² and/or R⁴. Therefore, the fluorine-containing ether compound represented by Formula (1) easily wets and spreads on the protective layer, and a lubricating layer having a uniform film state can be formed.

In addition, the N,N-substituted amides in Formulae (2) and (3) exhibit moderate interaction with the protective layer. Therefore, the N,N-substituted amide represented by Formulae (2) and (3), the polar group included in the divalent linking group represented by R² and/or R⁴ adjacent thereto, and the hydroxy group formed at one end of these groups in a case where one of R¹ or R⁵ is a hydrogen atom are all likely to be involved in bonding with the active point on the protective layer. As a result, the fluorine-containing ether compound represented by Formula (1) can form a lubricating layer having moderate and satisfactory adhesion to the protective layer.

Furthermore, the N,N-substituted amides represented by Formulae (2) and (3) have satisfactory fat-solubility.

From these, the end group represented by Formulae (2) and (3) has a function of forming a lubricating layer having satisfactory chemical substance resistance, wear resistance, and corrosion resistance in the fluorine-containing ether compound including the end group.

The end group represented by Formula (2) is a group in which a nitrogen atom constituting an amide bond of an N,N-substituted amide is bonded to a carbon atom of X¹. The end group represented by Formula (3) is a group in which a carbonyl carbon atom constituting an amide bond of an N,N-substituted amide is bonded to a carbon atom of X².

X¹ in the end group represented by Formula (2) and X² in the end group represented by Formula (3) are each an alkylene group having 1 to 30 carbon atoms. Xt (X²) may have a linear structure or a branched structure.

Since X¹ (X²) is an alkylene group having 1 or more carbon atoms, the N,N-substituted amide of Formulae (2) and (3) and the polar group of the divalent linking group, represented by R² and/or R⁴, adjacent to the N,N-substituted amide can be prevented from interacting with each other. Therefore, the N,N′-substituted amides represented by Formulae (2) and (3) and the polar group in the divalent linking group represented by R² and/or R⁴ adjacent to the N,N′-substituted amides can be involved in the bonding to the active point on the protective layer.

X¹ in the end group represented by Formula (2) is preferably an alkylene group having 2 or more carbon atoms. This is because, by spacing the nitrogen atom constituting the amide bond of the N,N′-substituted amide represented by Formula (2) and the polar group of the divalent linking group represented by R² and/or R⁴ adjacent to the end group from each other, the interaction between the N,N′-substituted amide represented by Formula (2) and the polar group of the divalent linking group represented by R² and/or R⁴ adjacent to the end group can be suppressed.

In addition, since X¹ (X²) is an alkylene group having 30 or less carbon atoms, the end group represented by Formula (2) (Formula (3)) in the fluorine-containing ether compound represented by Formula (1) does not cause steric hindrance that inhibits interaction with the protective layer. Therefore, the N,N-substituted amide of Formula (2) (Formula (3)) in the fluorine-containing ether compound provides a satisfactory affinity for the protective layer. X¹ (X²) is preferably a linear alkylene group having 6 or less carbon atoms, more preferably —CH₂—, —CH₂CH₂—, or —CH₂CH₂CH₂—, and still more preferably —CH₂— or —CH₂CH₂—. X¹ in the end group represented by Formula (2) is most preferably —CH₂CH₂—. In addition, it is most preferable that X² in the end group represented by Formula (3) is —CH₂—.

Y and Z in the end group represented by Formula (2) and A and B in the end group represented by Formula (3) are each independently an aliphatic group having 1 to 30 carbon atoms, which may include a polar group or an ether oxygen atom. The aliphatic group having 1 to 30 carbon atoms may be a saturated aliphatic group or an unsaturated aliphatic group.

Y and Z in the end group represented by Formula (2) may be bonded to each other to form a cyclic structure. A and B in the end group represented by Formula (3) may be bonded to each other to form a cyclic structure.

Examples of the polar group in a case where Y and Z in the end group represented by Formula (2) and A and B in the end group represented by Formula (3) have a polar group include a hydroxy group, a cyano group, an amino group, a carboxy group, a formyl group, a carbonyl group, and a sulfo group.

In Formula (2), one aliphatic group (Y in Formula (2)) having 1 to 30 carbon atoms, which may include a polar group or an ether oxygen atom, is bonded to a nitrogen atom constituting the amide bond of the N,N-substituted amide. In addition, in Formula (3), two aliphatic groups (A and B in Formula (3)) having 1 to 30 carbon atoms, which may include a polar group or an ether oxygen atom, are bonded to nitrogen atoms constituting the amide bond of the N,N-substituted amide.

Therefore, for example, the end group represented by Formula (2) (Formula (3)) has low hydrophilicity and has excellent fat-solubility as compared with a structure in which a hydrogen atom is bonded to Y in Formula (2) or a structure in which a hydrogen atom is bonded to at least one of A or B in Formula (3). Therefore, the fluorine-containing ether compound represented by Formula (1) can form a lubricating layer in which attraction and penetration of water can be suppressed and corrosion of a magnetic recording medium can be effectively suppressed as compared with a fluorine-containing ether compound including a structure in which a hydrogen atom is bonded instead of Y in Formula (2) or a structure in which a hydrogen atom is bonded instead of at least one of A or B in Formula (3).

In addition, the aliphatic group (Y in Formula (2)) bonded to the nitrogen atom constituting the amide bond in Formula (2) and the aliphatic groups (A and B in Formula (3)) bonded to the nitrogen atom constituting the amide bond in Formula (3) have a bulky structure as compared with a hydrogen atom. Therefore, the fluorine-containing ether compound represented by Formula (1) has a structure in which a hydrogen atom is bonded to Y in Formula (2) or a structure in which a hydrogen atom is bonded to at least one of A or B in Formula (3), and thus, the N,N-substituted amide of Formula (2) and Formula (3) and the polar group of the divalent linking group represented by R² and/or R⁴ adjacent thereto are unlikely to interact with each other, and thus, the fluorine-containing ether compound is unlikely to aggregate.

In a case where Y and Z in Formula (2) and A and B in Formula (3) are linear aliphatic amides not forming a cyclic structure with each other, the end group represented by Formula (2) or Formula (3) has high fluidity. Therefore, the fluorine-containing ether compound can form a lubricating layer having high restoring force and more excellent wear resistance.

In a case where Y and Z in Formula (2) and A and B in Formula (3) are linear aliphatic amides in which a cyclic structure is not formed between each other, the fluidity of the end groups represented by Formula (2) and Formula (3) is higher. Therefore, Y and Z in Formula (2) and A and B in Formula (3) are each independently preferably an aliphatic group having 1 to 8 carbon atoms, which may include a polar group or an ether oxygen atom, and more preferably an aliphatic group having 1 or 2 carbon atoms, which may include a polar group or an ether oxygen atom.

In a case where Y and Z in Formula (2) and A and B in Formula (3) do not form a cyclic structure with each other, specifically, it is preferable that Y and Z are each independently a methyl group, an ethyl group, a propyl group, a butyl group, a pentyl group, a hexyl group, an octyl group, an isopropyl group, a tertiary butyl group, an isoamyl group, a 1-hydroxyethyl group, a 1-hydroxypropyl group, a 2-hydroxypropyl group, a 1-methoxyethyl group, a 1-methoxypropyl group, a 2-methoxypropyl group, a vinyl group, an allyl group, a butenyl group, a propynyl group, a propargyl group, a butynyl group, a methylbutynyl group, a pentynyl group, a methylpentynyl group, a hexynyl group, or a cyanoethyl group. Among these, in a case where Y and Z in Formula (2) and A and B in Formula (3) do not form a cyclic structure with each other, Y and Z are preferably each independently a methyl group, an ethyl group, or a group selected from an allyl group, and more preferably each independently a methyl group or an ethyl group.

In a case where Y and Z in Formula (2) and A and B in Formula (3) are aliphatic amides which form a cyclic structure with each other, the lubricating layer containing this has suppressed hydrophilicity and has more excellent anticorrosion properties. In addition, in a case where Y and Z in Formula (2) and A and B in Formula (3) are aliphatic amides having a cyclic structure not including an ether oxygen atom, the lubricating layer including this has further suppressed hydrophilicity and has more excellent anticorrosion properties.

In a case where Y and Z in the end group represented by Formula (2) are an aliphatic amide in which a cyclic structure is formed between Y and Z, -Y-Z- in Formula (2) can be, for example, a combination of groups selected from the group consisting of a methylene group (—CH₂—), an ether bond (—O—), and an amine structure (—NH—). In a case where Y and Z in Formula (2) each form a cyclic structure, the cyclic structure is preferably a 5- to 7-membered ring including a carbonyl carbon atom and a nitrogen atom constituting an amide bond.

In addition, in a case where A and B in the end group represented by Formula (3) are an aliphatic amide in which a cyclic structure is formed between A and B, -A-B- in Formula (3) can be, for example, a combination of groups selected from the group consisting of a methylene group (—CH₂—), an ether bond (—O—), and an amine structure (—NH—). The cyclic structure in a case where A and B in Formula (3) form a cyclic structure with each other is preferably a 5- to 7-membered ring including a nitrogen atom constituting an amide bond.

-Y-Z- in Formula (2) and -A-B- in Formula (3) may have a polar group. In a case where -Y-Z- in Formula (2) and/or -A-B- in Formula (3) has a polar group, the polar group may be bonded to any carbon atom constituting the cyclic structure, and a polar group (for example, —NH—) may be included between the carbon atoms constituting the cyclic structure.

Examples of the end group represented by Formula (2) include any of the following organic groups represented by Formulae (2-1) to (2-5). In addition, examples of the end group represented by Formula (3) include any of the following organic groups represented by Formulae (3-1) to (3-5). The dotted line in Formulae (2-1) to (2-5) and Formulae (3-1) to (3-5) is a bonding site bonded to the oxygen atom of —R²—O— or —R⁴—O— in Formula (1).

The end group represented by Formula (2) or (3) in the fluorine-containing ether compound of the present embodiment is not limited to the organic groups shown in Formulae (2-1) to (2-5) and Formulae (3-1) to (3-5).

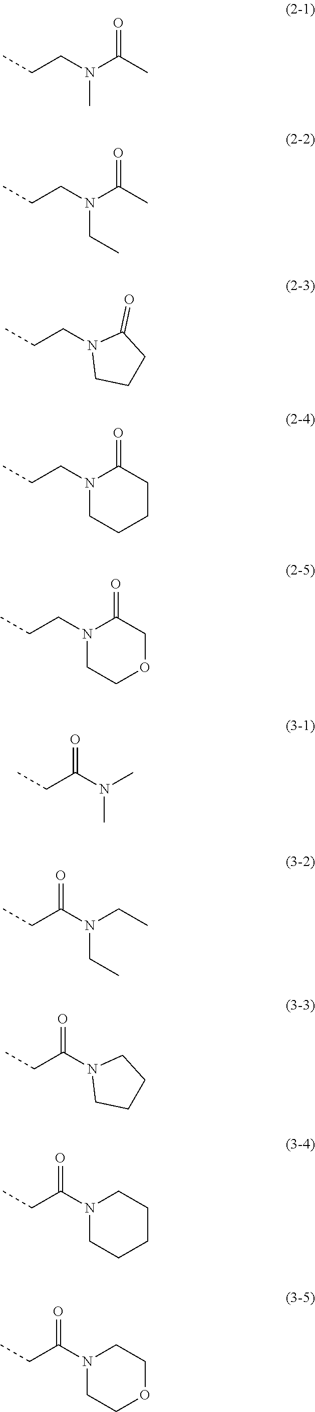

The organic group represented by Formula (2-1), (2-2), (3-1), or (3-2) is a linear aliphatic amide. Therefore, the organic group represented by Formula (2-1), (2-2), (3-1), or (3-2) has higher fluidity than the organic group represented by Formulae (2-3) to (2-5) or (3-3) to (3-5), which is an aliphatic amide having a cyclic structure. From this, in the lubricating layer containing the fluorine-containing ether compound in which R¹ and/or R⁵ is the organic group represented by Formula (2-1), (2-2), (3-1), or (3-2), even in a case where a part of the lubricating layer is deformed by abrasion and the fluorine-containing ether compound in the lubricating layer is moved to another location, a restoring force to return to the original position is high. Therefore, the fluorine-containing ether compound in which R¹ and/or R⁵ is the organic group represented by Formula (2-1), (2-2), (3-1), or (3-2) can form a lubricating layer having more excellent wear resistance.

The organic group represented by Formula (2-3), (2-4), (3-3), or (3-4) is an aliphatic amide having a cyclic structure, and does not include an ether oxygen atom. Therefore, the organic group represented by Formula (2-3), (2-4), (3-3), or (3-4) has satisfactory hydrophobicity as compared with the organic group represented by Formula (2-1), (2-2), (2-5), (3-1), (3-2), or (3-5). Therefore, the lubricating layer containing the fluorine-containing ether compound in which R¹ and/or R⁵ is the organic group represented by Formulae (2-3) to (2-4) or (3-3) to (3-4) can suppress the hydrophilicity to be low and the induction of water, which causes the corrosion of the magnetic recording medium. As a result, the fluorine-containing ether compound in which R¹ and/or R⁵ is the organic group represented by Formulae (2-3) to (2-4) or (3-3) to (3-4) can form a lubricating layer having a higher effect of suppressing corrosion of the magnetic recording medium.

The organic group represented by Formula (2-5) or (3-5) is an aliphatic amide having a cyclic structure including an ether oxygen atom. Therefore, the organic group represented by Formula (2-5) or (3-5) has higher flexibility than the organic group represented by Formula (2-1) to (2-4) or Formula (3-1) to (3-4). Therefore, the lubricating layer containing the fluorine-containing ether compound in which R¹ and/or R⁵ is the organic group represented by Formula (2-5) or (3-5) has moderate flexibility, and has a satisfactory affinity between the polar group in the fluorine-containing ether compound and the protective layer.

### (PFPE Chain Represented by R³)

In the fluorine-containing ether compound represented by Formula (1), R³ is a perfluoropolyether chain. In a case where the lubricant containing the fluorine-containing ether compound according to the present embodiment is applied onto the protective layer to form a lubricating layer, the PFPE chain represented by R³ coats a surface of the protective layer and imparts lubricity to the lubricating layer, thereby reducing a frictional force between the magnetic head and the protective layer. The PFPE chain represented by R³ is appropriately selected according to the performance required for the lubricant containing the fluorine-containing ether compound.

Examples of the PFPE chain represented by R³ include a polymerized substance or a copolymer of perfluoroalkylene oxide. Examples of the perfluoroalkylene oxide include perfluoromethylene oxide, perfluoroethylene oxide, perfluoro-n-propylene oxide, perfluoroisopropylene oxide, and perfluorobutylene oxide.

R³ in Formula (1) is preferably a PFPE chain represented by the following Formula (6) derived from, for example, a polymer or a copolymer of perfluoroalkylene oxide.

—(CF₂)_{w1}—O—(CF₂O)_{w2}—(CF₂CF₂O)_{w3}—(CF₂CF₂CF₂O)_{w4}—(CF₂CF₂CF₂CF₂O)_{w5}—(CF₂)_{w6}— (6)

(In Formula (6), w2, w3, w4, and w5 represent an average degree of polymerization, and each independently represent 0 to 20. Here, all of w2, w3, w4, and w5 do not represent 0 at the same time. w1 and w6 represent an average value representing the number of CF₂'s, and each independently represent 1 to 3. An arrangement order of (CF₂O), (CF₂CF₂O), (CF₂CF₂CF₂O), and (CF₂CF₂CF₂CF₂O), which are repeating units in Formula (6), is not particularly limited.)

In Formula (6), w2, w3, w4, and w5 represent an average degree of polymerization, and each independently represent 0 to 20, preferably 0 to 15 and more preferably 0 to 10.

In Formula (6), w1 and w6 are average values indicating the number of CF₂'s, and each independently represent 1 to 3. w1 and w6 are determined according to the structure of the repeating unit disposed at the end part of the chain structure in the PFPE chain represented by Formula (6) or the like.

(CF₂O), (CF₂CF₂O), (CF₂CF₂CF₂O), and (CF₂CF₂CF₂CF₂O) in Formula (6) are repeating units. The arrangement order of the repeating units in Formula (6) is not particularly limited. In addition, the number of kinds of repeating units in Formula (6) is also not particularly limited.

R³ in Formula (1) is preferably any one selected from the PFPE chains represented by Formulae (7-1) to (7-4).

In a case where R³ is any one selected from the PFPE chains represented by Formulae (7-1) to (7-4), the fluorine-containing ether compound is a compound having a lubricating layer having satisfactory lubricity. In addition, in a case where R³ is any one selected from the PFPE chains represented by Formulae (7-1) to (7-4), a proportion of the number of oxygen atoms (number of ether bonds (—O—)) to the number of carbon atoms in the PFPE chain is appropriate. Therefore, the fluorine-containing ether compound has moderate hardness. Therefore, the fluorine-containing ether compound applied onto the protective layer is unlikely to be aggregated on the protective layer, and a lubricating layer having a thinner thickness can be formed with a sufficient coating rate. In addition, the lubricating layer containing the fluorine-containing ether compound which is any one selected from the PFPE chains represented by Formulae (7-1) to (7-4) in which R³ is, is preferable because the lubricating layer is more dense.

—CF₂—(OCF₂CF₂)ₕ—(OCF₂)ᵢ—OCF₂— (7-1)

(In Formula (7-1), h and i represent an average degree of polymerization, where h represents 1 to 20 and i represents 0 to 20.)

—CF₂CF₂—(OCF₂CF₂CF₂)ⱼ—OCF₂CF₂— (7-2)

(In Formula (7-2), j represents an average degree of polymerization, and represents 1 to 15.)

—CF₂CF₂CF₂—(OCF₂CF₂CF₂CF₂)—OCF₂CF₂CF₂— (7-3)

(In Formula (7-3), k represents an average degree of polymerization, and represents 1 to 10.)

—(CF₂)_{w7}—O—(CF₂CF₂CF₂O)_{w5}—(CF₂CF₂O)_{w9}—(CF₂)_{w10}— (7-4)

(In Formula (7-4), w8 and w9 represent an average degree of polymerization, and each independently represents 1 to 20. w7 and w10 represent an average value representing the number of CF₂'s, and each independently represent 1 to 2.)

In Formula (7-1), the arrangement order of (OCF₂CF₂) and (OCF₂) which are the repeating units is not particularly limited. In Formula (7-1), the number h of (OCF₂CF₂)'s and the number i of (OCF₂)'s may be the same as or different from each other. The PFPE chain represented by Formula (7-1) may be a polymer of (OCF₂CF₂). In addition, the PFPE chain represented by Formula (7-1) may be any of a random copolymer, a block copolymer, or an alternating copolymer consisting of (OCF₂CF₂) and (OCF₂).

In Formulae (7-1) to (7-3), since h indicating the average degree of polymerization is 1 to 20, i is 0 to 20, j is 1 to 15, and k is 1 to 10, the lubricating layer having satisfactory lubricity is obtained. In addition, in Formulae (7-1) to (7-3), since h and i indicating the average degree of polymerization are 20 or less, j is 15 or less, and k is 10 or less, the viscosity of the fluorine-containing ether compound is not too high, and a lubricant containing the fluorine-containing ether compound is easily applied, which is preferable. h, i, j, and k indicating the average degree of polymerization represent preferably 1 to 10, more preferably 1.5 to 8, and still more preferably 2 to 7 from the viewpoint that the fluorine-containing ether compound that is easily wet and spreads on the protective layer and easily obtains a lubricating layer having a uniform film thickness is obtained.

In Formula (7-4), the arrangement order of (CF₂CF₂CF₂O) and (CF₂CF₂O), which are the repeating units, is not particularly limited. In Formula (7-4), the number w8 of (CF₂CF₂CF₂O)'s indicating the average degree of polymerization and the number w9 of (CF₂CF₂O)'s may be the same as or different from each other. Formula (7-4) may represent a random copolymer, a block copolymer, or an alternating copolymer including any of a random copolymer, a block copolymer, or an alternating copolymer consisting of a monomer unit (CF₂CF₂CF₂O) and (CF₂CF₂O).

In Formula (7-4), w8 and w9 representing an average degree of polymerization are each independently 1 to 20, preferably 1 to 15 and more preferably 1 to 10. w7 and w10 in Formula (7-4) are average values indicating the number of CF₂'s, and each independently represent 1 to 2. w7 and w10 are determined according to the structure of the repeating unit disposed at the end part of the chain structure in the PFPE chain represented by Formula (7-4) or the like.

The fluorine-containing ether compound represented by Formula (1) preferably has 4 to 6 polar groups. The above-described polar group also includes N,N-substituted amide of an end group represented by Formula (2) or (3).

In a case where the number of polar groups in the fluorine-containing ether compound represented by Formula (1) is 4 or more, the adhesion of the lubricating layer containing the protective layer is further improved, and a lubricating layer having a satisfactory coating state is easily obtained. In addition, in a case where the number of polar groups included in the fluorine-containing ether compound represented by Formula (1) is 6 or less, the hydrophilicity of the fluorine-containing ether compound represented by Formula (1) is too high, and thus it is possible to suppress the generation of water, which causes corrosion, in the lubricating layer containing the fluorine-containing ether compound. Therefore, the fluorine-containing ether compound represented by Formula (1) in which the number of polar groups is 6 or less can form a lubricating layer having a higher corrosion suppression effect on the magnetic recording medium.

In the fluorine-containing ether compound represented by Formula (1), R¹—O—R²— and —R⁴—O—R⁵— may be the same as or different from each other. It is preferable that R¹—O—R²— and —R⁴—O—R⁵ are the same as each other from the viewpoint that the fluorine-containing ether compound is easily synthesized.

Specifically, the fluorine-containing ether compound represented by Formula (1) is preferably any of compounds represented by Formulae (AA) to (AL) and (BA) to (BF).

In a case where the compound represented by Formula (1) is any of compounds represented by Formulae (AA) to (AL) and (BA) to (BF), a raw material is easily available, and a lubricating layer having satisfactory chemical substance resistance and wear resistance and a high corrosion suppression effect on the magnetic recording medium can be formed even in a case where the thickness is small.

In the compounds represented by Formulae (AA) to (AL) and (BA) to (BF), Rf₁, Rf₂, and Rf₃ representing a PFPE chain each have the following structure. That is, in the compounds represented by Formulae (AA) to (AL), and (BA) to (BD), Rf₁ is the PFPE chain represented by Formula (7-1). In the compound represented by Formula (BE), Rf₂ is the PFPE chain represented by Formula (7-2). In the compound represented by Formula (BF), Rf₁ is the PFPE chain represented by Formula (7-3). In Formulae (AA) to (AL) and (BA) to (BF), h and i in Rf₁ representing the PFPE chain, j in Rf₂, and k in Rf₃ are values indicating an average degree of polymerization, and thus are not always integers.

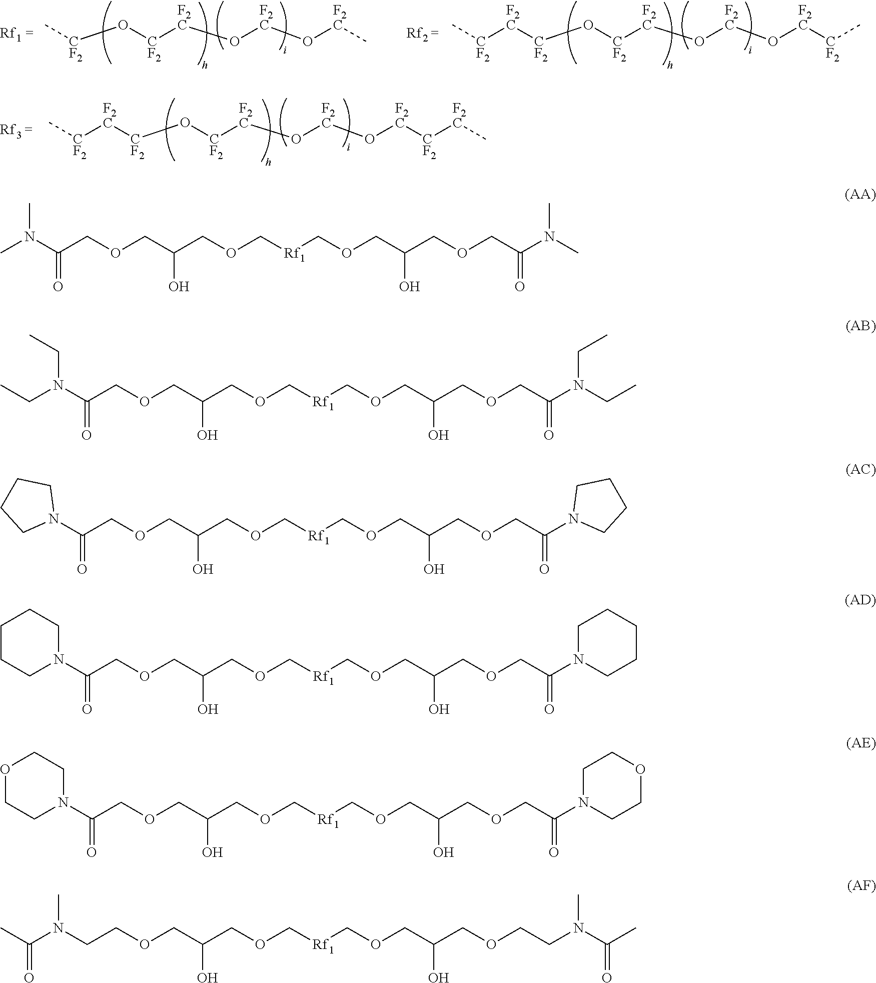

(In Rf₁ in Formula (AA), h and i represent an average degree of polymerization, where h represents 1 to 20 and i represents 0 to 20.)

(In Rf₁ in Formula (AB), h and i represent an average degree of polymerization, where h represents 1 to 20 and i represents 0 to 20.)

(In Rf₁ in Formula (AC), h and i represent an average degree of polymerization, where h represents 1 to 20 and i represents 0 to 20.)

(In Rf₁ in Formula (AD), h and i represent an average degree of polymerization, where h represents 1 to 20 and i represents 0 to 20.)

(In Rf₁ in Formula (AE), h and i represent an average degree of polymerization, where h represents 1 to 20 and i represents 0 to 20.)

(In Rf₁ in Formula (AF), h and i represent an average degree of polymerization, where h represents 1 to 20 and i represents 0 to 20.)

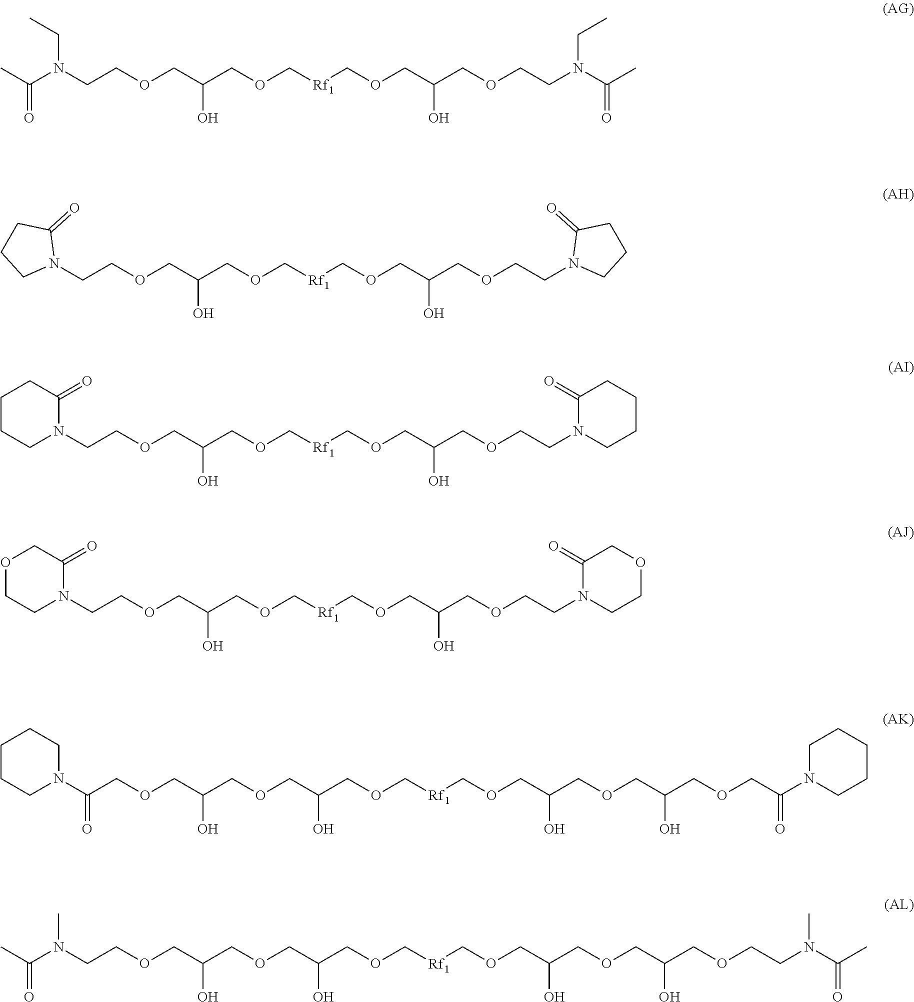

(In Rf₁ in Formula (AG), h and i represent an average degree of polymerization, where h represents 1 to 20 and i represents 0 to 20.)

(In Rf₁ in Formula (AH), h and i represent an average degree of polymerization, h represents 1 to 20, and i represents 0 to 20.)

(In Rf₁ in Formula (AI), h and i represent an average degree of polymerization, where h represents 1 to 20 and i represents 0 to 20.)

(In Rf₁ in Formula (AJ), h and i represent an average degree of polymerization, where h represents 1 to 20 and i represents 0 to 20.)

(In Rf₁ in Formula (AK), h and i represent an average degree of polymerization, where h represents 1 to 20 and i represents 0 to 20.)

(In Rf₁ in Formula (AL), h and i represent an average degree of polymerization, where h represents 1 to 20 and i represents 0 to 20.)

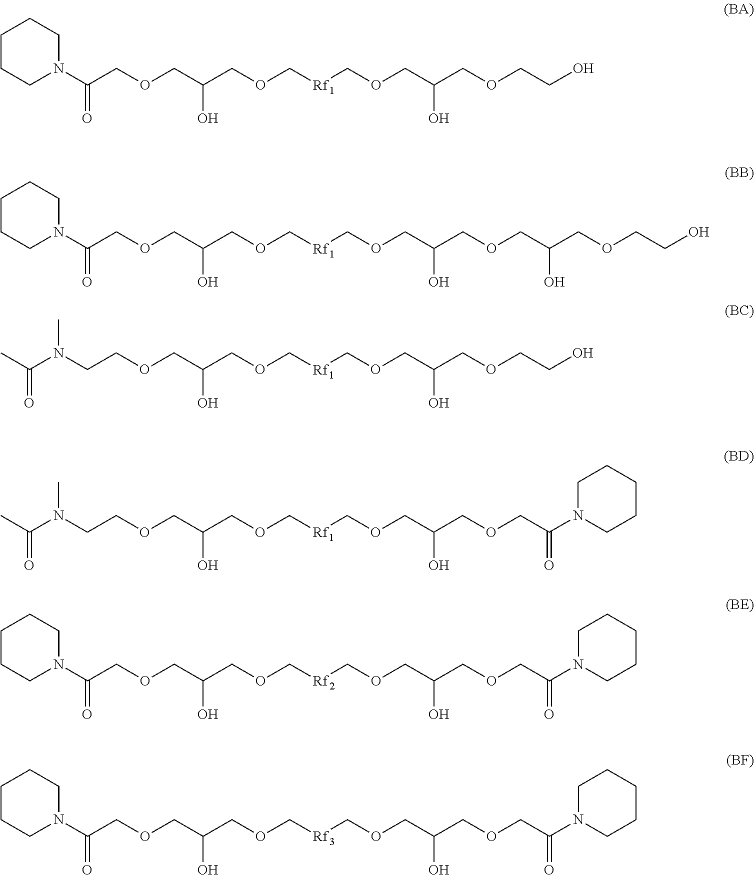

(In Rf₁ of Formula (BA), h and i represent an average degree of polymerization, where h represents 1 to 20 and i represents 0 to 20.)

(In Rf₁ in Formula (BB), h and i represent an average degree of polymerization, where h represents 1 to 20 and i represents 0 to 20.)

(In Rf₁ in Formula (BC), h and i represent an average degree of polymerization, where h represents 1 to 20 and i represents 0 to 20.)

(In Rf₁ in Formula (BD), h and i represent an average degree of polymerization, where h represents 1 to 20 and i represents 0 to 20.)

(In Rf₂ in Formula (BE), j represents an average degree of polymerization, and represents 1 to 15.)

(In Rf₃ in Formula (BF), k represents an average degree of polymerization and represents 1 to 10.)

The number-average molecular weight (Mn) of the fluorine-containing ether compound of the present embodiment is preferably in a range of 500 to 10,000 and particularly preferably in a range of 1,000 to 5,000. In a case where the number-average molecular weight thereof is 500 or greater, the lubricating layer formed of the lubricant containing the fluorine-containing ether compound according to the present embodiment has excellent heat resistance. The number-average molecular weight of the fluorine-containing ether compound is more preferably 1,000 or greater. In addition, in a case where the number-average molecular weight thereof is 10,000 or less, the viscosity of the fluorine-containing ether compound is appropriate, and a lubricating layer with a small film thickness can be easily formed by applying a lubricant containing the fluorine-containing ether compound. The number-average molecular weight of the fluorine-containing ether compound is preferably 5,000 or less from the viewpoint that the viscosity is easy to handle in a case where the fluorine-containing ether compound is applied to a lubricant.

The number-average molecular weight (Mn) of the fluorine-containing ether compound is a value measured by ¹H-NMR and ¹⁹F-NMR using AVANCE III 400 (manufactured by Bruker BioSpin GmbH). Specifically, the number of repeating units of the PFPE chain is calculated from the integrated value measured by ¹⁹F-NMR. and the number-average molecular weight is determined. In the measurement of nuclear magnetic resonance (NMR), the sample is diluted with a solvent of hexafluorobenzene/d-acetone (4/1 v/v) and measured. The peak of hexafluorobenzene is set to −164.7 ppm as the reference of the ¹⁹F-NMR chemical shift, and the peak of acetone is set to 2.2 ppm as the reference of the ¹H-NMR chemical shift.

In the fluorine-containing ether compound of the present embodiment, it is preferable that the polydispersity (ratio of weight-average molecular weight (Mw) to number-average molecular weight (Mn)) is set to 1.3 or less by fractionating the molecular weight by an appropriate method.

In the present embodiment, a method for fractionating the molecular weight is not particularly limited, and for example, molecular weight fractionation by a silica gel column chromatography method, a gel permeation chromatography (GPC) method, or the like, molecular weight fractionation by a supercritical extraction method, and the like can be used.

### “Production Method”

A method for producing the fluorine-containing ether compound of the present embodiment is not particularly limited, and the fluorine-containing ether compound can be produced by a known production method of the related art. The fluorine-containing ether compound of the present embodiment can be produced, for example, by the following production method.

(In case where R¹—O—R²— and —R⁴—O—R⁵ are the same as each other)

In Formula (1), in order to produce a compound in which R¹—O—R²— and —R⁴—O—R⁵— are the same, first, a fluorine-based compound in which hydroxymethyl groups (—CH₂OH) are disposed at both ends of a perfluoropolyether chain corresponding to R³ in Formula (1) is prepared.

Next, the hydroxy group of the hydroxymethyl group disposed at one end of the fluorine-based compound is reacted with the epoxy group of the epoxy compound having the group as R¹—O—R²— (group represented by ═—R⁴—O—R⁵) represented by Formula (1). Accordingly, the fluorine-containing ether compound of the present embodiment, in which both ends of the perfluoropolyether chain corresponding to R³ have the group corresponding to R¹—O—R²— (group corresponding to =—R⁴—O—R¹—)

As the epoxy compound having a group represented by R¹—O—R²— in Formula (1) (group represented by ═—R⁴—O—R⁵—), for example, a compound represented by Formulae (8-1) to (8-14) can be used. In Formulae (8-11) to (8-14), THP represents a tetrahydropyranyl group.

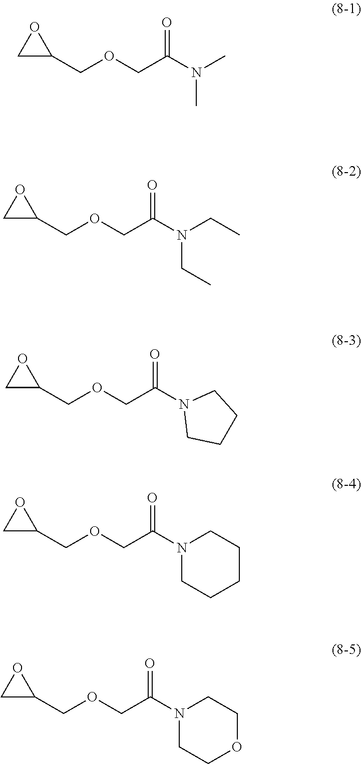

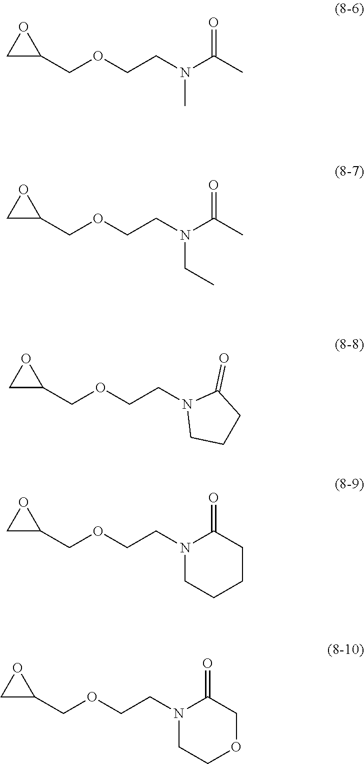

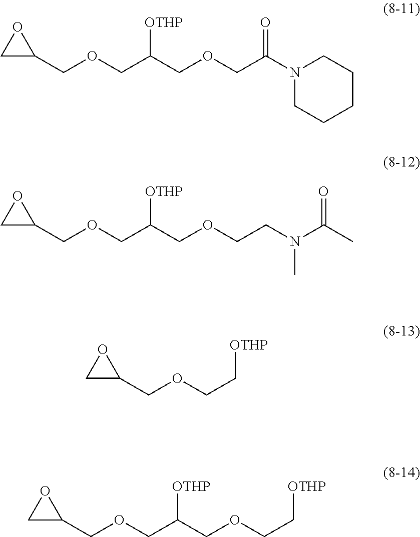

In a case where the fluorine-based compound and the epoxy compound react with each other, the hydroxy group of the epoxy compound may be protected with an appropriate protective group, and then the fluorine-based compound may react with the epoxy compound.

(In case where R¹—O—R²— and —R⁴—O—R⁵ are different from each other)

In Formula (1), in order to produce a compound in which R¹—O—R²— and —R⁴—O— R⁵— are different from each other, first, a fluorine-based compound in which hydroxymethyl groups (—CH₂OH) are disposed at both ends of the perfluoropolyether chain corresponding to R³ in Formula (1) is prepared.

Next, a hydroxy group of the hydroxymethyl group disposed at one end of the fluorine-based compound and an epoxy group of the epoxy compound having a group represented by R¹—O—R²— in Formula (1) are reacted with each other. In this manner, an intermediate compound 1 having a group corresponding to R¹—O—R²— at one end of the perfluoropolyether chain corresponding to R³ is obtained (first reaction).

Next, the above-described intermediate compound 1 and the epoxy compound having a group represented by —R⁴—O—R⁵ in Formula (1) are reacted with each other (second reaction).

By performing the above-described steps, the fluorine-containing ether compound of the present embodiment, in which R¹—O—R²— and —R⁴—O—R⁵ in Formula (1) are different from each other, is obtained.

### [Lubricant for Magnetic Recording Medium]

A lubricant for a magnetic recording medium according to the present embodiment contains the fluorine-containing ether compound represented by Formula (1).

In the lubricant of the present embodiment, as long as the characteristics are not impaired by containing the fluorine-containing ether compound represented by Formula (1), known materials used as a material for the lubricant can be used in the form of a mixture as necessary.

Specific examples of the known materials include, for example, FOMBLIN (registered trademark) ZDIAC, FOMBLIN ZDEAL, and FOMBLIN AM-2001 (all manufactured by Solvay Solexis), and Moresco A20H (manufactured by Moresco). The known materials used in the form of a mixture with the lubricant according to the present embodiment has a number-average molecular weight of preferably 1000 to 10000.

In a case where the lubricant of the present embodiment contains other materials of the fluorine-containing ether compound represented by Formula (1), the content of the fluorine-containing ether compound represented by Formula (1) in the lubricant of the present embodiment is preferably 70% by mass or greater, more preferably 90% by mass or greater, and still more preferably 95% by mass or greater.

Since the lubricant of the present embodiment contains the fluorine-containing ether compound represented by Formula (1), the lubricant can form a lubricating layer having satisfactory chemical substance resistance and wear resistance and a high corrosion suppression effect on the magnetic recording medium.

### [Magnetic Recording Medium]

The magnetic recording medium according to the present embodiment is formed by sequentially providing at least a magnetic layer, a protective layer, and a lubricating layer on a substrate.

In the magnetic recording medium according to the present embodiment, one or two or more underlayers can be provided between the substrate and the magnetic layer as necessary. In addition, at least one of an adhesive layer or a soft magnetic layer can be provided between the underlayer and the substrate.

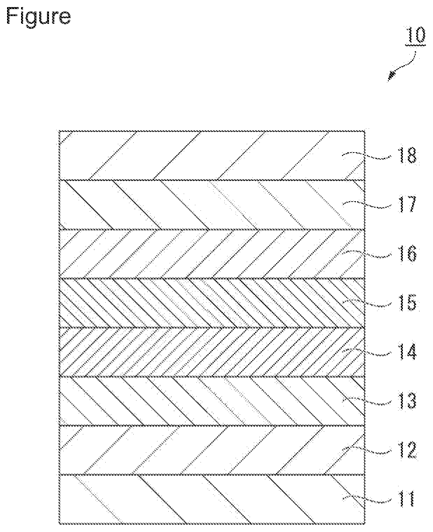

FIG. 1 is a schematic cross-sectional view showing an embodiment of the magnetic recording medium according to the present invention.

The magnetic recording medium 10 according to the present embodiment has a structure in which an adhesive layer 12, a soft magnetic layer 13, a first underlayer 14, a second underlayer 15, a magnetic layer 16, a protective layer 17, and a lubricating layer 18 are sequentially provided on a substrate 11.

### “Substrate”

As the substrate 11, for example, a non-magnetic substrate on which a film made of NiP or a NiP alloy is formed on a base made of a metal or an alloy material such as Al or an Al alloy can be used.

In addition, as the substrate 11, a non-magnetic substrate made of a non-metal material such as glass, ceramics, silicon, silicon carbide, carbon, or a resin may be used, or a non-magnetic substrate in which a film of NiP or a NiP alloy is formed on a base made of these non-metal materials may be used.

### “Adhesive Layer”

The adhesive layer 12 prevents the progression of corrosion of the substrate 11 in a case where the substrate 11 and the soft magnetic layer 13 provided on the adhesive layer 12 are disposed in contact with each other.

The material of the adhesive layer 12 can be appropriately selected from, for example, Cr, a Cr alloy, Ti, a Ti alloy, CrTi, NiAl, and an AIRu alloy. The adhesive layer 12 can be formed by, for example, a sputtering method.

### “Soft Magnetic Layer”

It is preferable that the soft magnetic layer 13 has a structure in which a first soft magnetic film, an intermediate layer formed of a Ru film, and a second soft magnetic film are sequentially laminated. That is, it is preferable that the soft magnetic layer 13 has a structure in which the upper and lower soft magnetic films of the intermediate layer are antiferro-coupled (AFC) by interposing the intermediate layer formed of a Ru film between the two layers of the soft magnetic films.

Examples of the material of the first soft magnetic film and the second soft magnetic film include a CoZrTa alloy and a CoFe alloy.

It is preferable to add any of Zr, Ta, or Nb to the CoFe alloy used in the first soft magnetic film and the second soft magnetic film. In this manner, the amorphization of the first soft magnetic film and the second soft magnetic film is promoted. As a result, it is possible to improve the aligning properties of the first underlayer (seed layer) and to reduce the floating amount of the magnetic head.

The soft magnetic layer 13 can be formed by, for example, a sputtering method.

### “First Underlayer”

The first underlayer 14 is a layer that controls the alignment and the crystal size of the second underlayer 15 and the magnetic layer 16 provided thereon.

Examples of the first underlayer 14 include a Cr layer, a Ta layer, a Ru layer, a CrMo alloy layer, a CoW alloy layer, a CrW alloy layer, a CrV alloy layer, and a CrTi alloy layer.

The first underlayer 14 can be formed by, for example, a sputtering method.

### “Second Underlayer”

The second underlayer 15 is a layer in which the alignment of the magnetic layer 16 is controlled to be satisfactory. The second underlayer 15 is preferably a layer formed of Ru or a Ru alloy.

The second underlayer 15 may consist of a single layer or a plurality of layers. In a case where the second underlayer 15 consists of a plurality of layers, all the layers may be formed of the same material, or at least one layer may be formed of a different material.

The second underlayer 15 can be formed by, for example, a sputtering method.

### “Magnetic Layer”

The magnetic layer 16 is formed of a magnetic film in which a magnetization easy axis is oriented in a direction perpendicular or horizontal to the substrate surface. The magnetic layer 16 is a layer containing Co and Pt. The magnetic layer 16 may be a layer containing an oxide, Cr, B, Cu, Ta, Zr, or the like in order to improve the SNR characteristics.

Examples of the oxide contained in the magnetic layer 16 include SiO₂, SiO, Cr₂O₃, CoO, Ta₂O₃, and TiO₂.

The magnetic layer 16 may consist of one layer or a plurality of magnetic layers formed of materials having different compositions.

For example, in a case where the magnetic layer 16 consists of three layers of a first magnetic layer, a second magnetic layer, and a third magnetic layer which are laminated in this order from the bottom, it is preferable that the first magnetic layer has a granular structure formed of a material containing Co, Cr, and Pt and further containing an oxide. As the oxide contained in the first magnetic layer, for example, an oxide of Cr, Si, Ta, Al, Ti, Mg, or Co is preferably used. Among these, TiO₂, Cr₂O₃, SiO₂, and the like can be particularly suitably used. In addition, it is preferable that the first magnetic layer is formed of a composite oxide to which two or more kinds of oxides are added. Among these, Cr₂O₃—SiO₂, Cr₂O₃—TiO₂, SiO₂—TiO₂, or the like can be particularly suitably used.

The first magnetic layer can contain one or more kinds of elements selected from B, Ta, Mo, Cu, Nd, W, Nb, Sm, Tb, Ru, and Re in addition to Co, Cr, Pt, and an oxide.

The same material as that of the first magnetic layer can be used for the second magnetic layer. It is preferable that the second magnetic layer has a granular structure.

It is preferable that the third magnetic layer has a non-granular structure formed of a material containing Co, Cr, and Pt and not containing an oxide. The third magnetic layer can contain one or more elements selected from B, Ta, Mo, Cu, Nd, W, Nb, Sm, Tb, Ru, Re, and Mn in addition to Co, Cr, and Pt.

In a case where the magnetic layer 16 is formed of a plurality of magnetic layers, it is preferable to provide a non-magnetic layer between the adjacent magnetic layers. In a case where the magnetic layer 16 is formed of three layers of a first magnetic layer, a second magnetic layer, and a third magnetic layer, it is preferable to provide a non-magnetic layer between the first magnetic layer and the second magnetic layer and between the second magnetic layer and the third magnetic layer.

As the non-magnetic layer provided between adjacent magnetic layers of the magnetic layer 16, for example, Ru, a Ru alloy, a CoCr alloy, or a CoCrX1 alloy (X1 represents one or two or more kinds of elements selected from Pt, Ta, Zr, Re, Ru, Cu, Nb, Ni, Mn, Ge, Si, O, N, W, Mo, Ti, V, and B) can be suitably used.

It is preferable that, as the non-magnetic layer provided between adjacent magnetic layers of the magnetic layer 16, an alloy material containing an oxide, a metal nitride, or a metal carbide is used. Specifically, for example, SiO₂, Al₂O₃, Ta₂O₅, Cr₂O₃, MgO, Y₂O₃, or TiO₂ can be used as the oxide. As the metal nitride, for example, AlN, Si₃N₄, TaN, or CrN can be used. As the metal carbide, for example, TaC, BC, or SiC can be used.

The non-magnetic layer can be formed by, for example, a sputtering method.

In order to achieve a higher recording density, it is preferable that the magnetic layer 16 is a magnetic layer for perpendicular magnetic recording in which a magnetization easy axis is oriented in a direction perpendicular to the substrate surface. The magnetic layer 16 may be a magnetic layer for in-plane magnetic recording.

The magnetic layer 16 may be formed by any of the known methods in the related art, such as an evaporation method, an ion beam sputtering method, and a magnetron sputtering method. The magnetic layer 16 is usually formed by a sputtering method.

### “Protective Layer”

The protective layer 17 protects the magnetic layer 16. The protective layer 17 may be formed of one layer or a plurality of layers. As the protective layer 17, a carbon-based protective layer can be preferably used, and an amorphous carbon protective layer is particularly preferable. In a case where the protective layer 17 is a carbon-based protective layer, the interaction with the polar group (particularly, the hydroxy group) contained in the fluorine-containing ether compound in the lubricating layer 18 is further enhanced, which is preferable.

The adhesion force between the carbon-based protective layer and the lubricating layer 18 can be controlled by forming the carbon-based protective layer with hydrocarbon and/or nitrogenated carbon and adjusting the hydrogen content and/or the nitrogen content in the carbon-based protective layer. The hydrogen content in the carbon-based protective layer is preferably in a range of 3% by atom to 20% by atom in a case of being measured by a hydrogen forward scattering method (HFS). In addition, the nitrogen content in the carbon-based protective layer is preferably in a range of 4% by atom to 15% by atom in a case of being measured by X-ray photoelectron spectroscopy (XPS).

The hydrogen and/or nitrogen contained in the carbon-based protective layer are not necessarily uniformly contained in the entire carbon-based protective layer. It is suitable that the carbon-based protective layer is, for example, a composition gradient layer in which nitrogen is contained on the lubricating layer 18 side of the protective layer 17 and hydrogen is contained on the magnetic layer 16 side of the protective layer 17. In this case, the adhesion force between the magnetic layer 16, the lubricating layer 18, and the carbon-based protective layer is further improved.

The film thickness of the protective layer 17 is preferably in a range of 1 nm to 7 nm. In a case where the film thickness of the protective layer 17 is 1 nm or greater, the performance as the protective layer 17 can be sufficiently obtained. It is preferable that the film thickness of the protective layer 17 is 7 nm or less from the viewpoint of reducing the film thickness of the protective layer 17.

As a film forming method of the protective layer 17, a sputtering method using a target material containing carbon, a CVD (chemical vapor deposition) method using a hydrocarbon raw material such as ethylene or toluene, an ion beam deposition (IBD) method, or the like can be used.

In a case where the carbon-based protective layer is formed as the protective layer 17, the carbon-based protective layer can be formed, for example, by a DC magnetron sputtering method. In particular, in a case where a carbon-based protective layer is formed as the protective layer 17, it is preferable to form an amorphous carbon protective layer by a plasma CVD method. The amorphous carbon protective layer formed by the plasma CVD method has a uniform surface and small roughness.

### “Lubricating Layer”

The lubricating layer 18 prevents contamination of the magnetic recording medium 10. In addition, the lubricating layer 18 reduces a frictional force of the magnetic head of the magnetic recording and reproducing device that slides on the magnetic recording medium 10 to improve the durability of the magnetic recording medium 10.

As shown in FIG. 1, the lubricating layer 18 is formed in contact with the protective layer 17. The lubricating layer 18 is formed by applying the lubricant for a magnetic recording medium according to the embodiment described above onto the protective layer 17. Therefore, the lubricating layer 18 contains the above-described fluorine-containing ether compound.

In a case where the protective layer 17 disposed below the lubricating layer 18 is a carbon-based protective layer, the lubricating layer 18 is particularly bonded to the protective layer 17 with a high binding force. As a result, even in a case where the thickness of the lubricating layer 18 is small, the magnetic recording medium 10 in which the surface of the protective layer 17 is coated at a high coating rate is likely to be obtained, and the contamination of the surface of the magnetic recording medium 10 can be effectively prevented.

The average film thickness of the lubricating layer 18 is preferably in a range of 0.5 nm (5 Å) to 2.0 nm (20 Å) and more preferably in a range of 0.5 nm (5 Å) to 1.2 nm (12 Å). In a case where the average film thickness of the lubricating layer 18 is 0.5 nm or greater, the lubricating layer 18 is formed with a uniform film thickness without being island-like or mesh-like. Therefore, the surface of the protective layer 17 can be coated with the lubricating layer 18 at a high coating rate. In addition, in a case where the average film thickness of the lubricating layer 18 is set to 2.0 nm or less, the lubricating layer 18 can be sufficiently thinned, and the floating amount of the magnetic head can be sufficiently reduced.

### “Method for Forming Lubricating Layer”

Examples of a method of forming the lubricating layer 18 include a method of preparing a magnetic recording medium in the middle of production in which each layer up to the protective layer 17 is formed on the substrate 11, coating the protective layer 17 with a lubricating layer forming solution, and drying the solution.

The lubricating layer forming solution is obtained by dispersing and dissolving the lubricant for a magnetic recording medium according to the embodiment described above in a solvent as necessary, and setting the viscosity and the concentration suitable for a coating method.

Examples of a solvent used in the lubricating layer forming solution include a fluorine-based solvent such as VERTREL (registered trademark) XF (trade name, manufactured by DuPont Mitsui Fluorochemicals Co., Ltd.) or ASAHIKLIN (registered trademark) AE-3000 (trade name, manufactured by AGC Inc.).

A coating method for the lubricating layer forming solution is not particularly limited, and examples thereof include a spin coating method, a spraying method, a paper coating method, and a dipping method.

In a case where the dipping method is used, for example, the following method can be used. First, the substrate 11 on which each layer up to the protective layer 17 is formed is immersed in a lubricating layer forming solution placed in an immersion tank of a dip coating device. Next, the substrate 11 is pulled up from the immersion tank at a predetermined speed. In this manner, the lubricating layer forming solution is applied onto the surface of the protective layer 17 of the substrate 11.

By using the dipping method, the lubricating layer forming solution can be uniformly applied to the surface of the protective layer 17, and the lubricating layer 18 can be formed on the protective layer 17 with a uniform film thickness.

In the present embodiment, it is preferable to perform a thermal treatment on the substrate 11 on which the lubricating layer 18 has been formed. By performing the thermal treatment, the adhesion between the lubricating layer 18 and the protective layer 17 is improved, and the adhesion force between the lubricating layer 18 and the protective layer 17 is improved.

The thermal treatment temperature is preferably in a range of 100° C. to 180° C. and more preferably in a range of 100° C. to 160° C. In a case where the thermal treatment temperature is 100° C. or higher, the effect of improving the adhesion between the lubricating layer 18 and the protective layer 17 is sufficiently obtained. In addition, thermal decomposition of the lubricating layer 18 due to the thermal treatment can be prevented by setting the thermal treatment temperature to 180° C. or lower. The thermal treatment time can be appropriately adjusted according to the thermal treatment temperature and is preferably 10 minutes to 120 minutes.

In the present embodiment, in order to further improve the adhesion force of the lubricating layer 18 to the protective layer 17, a treatment of irradiating the lubricating layer 18 with ultraviolet rays (UV) may be performed before or after the thermal treatment.

The magnetic recording medium 10 according to the present embodiment is formed by sequentially providing at least the magnetic layer 16, the protective layer 17, and the lubricating layer 18 on the substrate 11. In the magnetic recording medium 10 according to the present embodiment, the lubricating layer 18 containing the above-described fluorine-containing ether compound is formed in contact with the protective layer 17. The lubricating layer 18 has satisfactory chemical substance resistance and wear resistance, and a high corrosion suppression effect of the magnetic recording medium even in a case where the film thickness thereof is small. Therefore, the magnetic recording medium 10 according to the present embodiment has excellent reliability, particularly excellent corrosion resistance and durability. Therefore, the magnetic recording medium 10 according to the present embodiment can contribute to the reduction of the magnetic spacing, and the floating amount of the magnetic head can be reduced (for example, 10 nm or less), and the magnetic recording medium 10 can stably operate for a long period of time even in a severe environment due to the diversification of applications. Therefore, the magnetic recording medium 10 according to the present embodiment is particularly suitable as a magnetic disk mounted on a magnetic disk apparatus of a load unload (LUL) system.

### EXAMPLES

Hereinafter, the present invention will be described in more detail with reference to examples and comparative examples. Further, the present invention is not limited to the following examples.

### Example 1

A compound represented by Formula (AA) was obtained by the following method.

A 100 mL eggplant flask was charged with 5 g of a compound represented by HOCH₂CF₂O(CF₂CF₂O)ₕ(CF₂O)ᵢCF₂CH₂OH (h representing the average degree of polymerization in the formula represent 4.5, and i representing the average degree of polymerization represent 4.5) (number-average molecular weight: 1,000, molecular weight distribution: 1.1), 3.18 g of a compound represented by Formula (8-1), and 5 mL of t-butanol in a nitrogen gas atmosphere, and the mixture was stirred at room temperature until the mixture was uniform. 0.30 g of potassium tert-butoxide was added to the mixture, and the mixture was stirred at 70° C. for 16 hours to carry out a reaction.

The compound represented by Formula (8-1) was synthesized by reacting a hydroxy group of N,N-dimethylglycolamide with epibromohydrin.

Thereafter, the reaction solution was gradually transferred to a separatory funnel containing 25 mL of saline, and extracted twice with 50 mL of ethyl acetate. The organic layer was washed with 25 mL of saline, 25 mL of saturated aqueous sodium bicarbonate, and 25 mL of saline in this order, and then dewatered with anhydrous sodium sulfate. The drying agent was separated by filtration, and the filtrate was concentrated, and the residues were purified by silica gel column chromatography, thereby obtaining 4.09 g of a compound (AA) (Rf₁ in Formula (AA) is a PFPE chain represented by Formula (7-1), and in Rf₁, h representing the average degree of polymerization represents 4.5, and i representing the average degree of polymerization represents 4.5.).

The structure of the obtained compound (AA) was identified by performing ¹H-NMR measurement and ¹⁹F-NMR measurement based on the following results.

¹H-NMR (acetone-D₆): δ [ppm]=3.25-3.60 (8H), 3.65-3.95 (4H), 3.75-4.00 (2H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 2

A compound represented by Formula (AB) was obtained by the following method.

4.15 g of a compound (AB) (Rf₁ in Formula (AB) is a PFPE chain represented by Formula (7-1), and in Rf₁, h representing the average degree of polymerization represents 4.5, and i representing the average degree of polymerization represents 4.5) was obtained by performing the same operation as in Example 1 except that a compound represented by Formula (8-2) was used instead of the compound represented by Formula (8-1).

The compound represented by Formula (8-2) was synthesized by reacting a hydroxy group of N,N-diethylglycolamide with epibromohydrin.

The structure of the obtained compound (AB) was identified by performing ¹H-NMR measurement and ¹⁹F-NMR measurement based on the following results.

¹H-NMR (acetone-D₆): δ [ppm]=1.20-1.30 (12H), 3.20-3.60 (12H), 3.65-3.95 (4H), 3.75-4.00 (2H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 3

A compound represented by Formula (AC) was obtained by the following method.

4.55 g of a compound (AC) (Rf₁ in Formula (AC) is a PFPE chain represented by Formula (7-1), and in Rf₁, h representing the average degree of polymerization represents 4.5, and i representing the average degree of polymerization represents 4.5) was obtained by performing the same operation as in Example 1 except that a compound represented by Formula (8-3) was used instead of the compound represented by Formula (8-1).

The compound represented by Formula (8-3) was synthesized by reacting a hydroxy group of glycolylpyrrolidine with epibromohydrin.

The structure of the obtained compound (AC) was identified by performing ¹H-NMR measurement and ¹⁹F-NMR measurement based on the following results.

¹H-NMR (acetone-D₆): δ [ppm]=1.95-2.15 (8H), 3.20-3.60 (12H), 3.65-3.95 (4H), 3.75-4.00 (2H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 4

A compound represented by Formula (AD) was obtained by the following method.

4.60 g of a compound (AD) (Rf₁ in Formula (AD) is a PFPE chain represented by Formula (7-1), and in Rf₁, h representing the average degree of polymerization represents 4.5, and i representing the average degree of polymerization represents 4.5) was obtained by performing the same operation as in Example 1 except that a compound represented by Formula (8-4) was used instead of the compound represented by Formula (8-1).

The compound represented by Formula (8-4) was synthesized by reacting a hydroxy group of glycolylpiperidine with epibromohydrin.

The structure of the obtained compound (AD) was identified by performing ¹H-NMR measurement and ¹⁹F-NMR measurement based on the following results.

¹H-NMR (acetone-D₆): δ [ppm]=1.30-1.45 (4H), 1.95-2.10 (8H), 3.20-3.65 (12H), 3.65-3.95 (4H), 3.75-4.00 (2H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 5

A compound represented by Formula (AE) was obtained by the following method.

4.55 g of a compound (AE) (Rf₁ in Formula (AE) is a PFPE chain represented by Formula (7-1), and in Rf₁, h representing the average degree of polymerization represents 4.5, and i representing the average degree of polymerization represents 4.5) was obtained by performing the same operation as in Example 1 except that a compound represented by Formula (8-5) was used instead of the compound represented by Formula (8-1).

The compound represented by Formula (8-5) was synthesized by reacting a hydroxy group of glycolylmorpholine with epibromohydrin.

The structure of the obtained compound (AE) was identified by performing ¹H-NMR measurement and ¹⁹F-NMR measurement based on the following results.

¹H-NMR (acetone-D₆): δ [ppm]=3.20-3.65 (20H), 3.65-3.95 (4H), 3.75-4.00 (2H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 6

A compound represented by Formula (AF) was obtained by the following method.

4.00 g of a compound (AF) (Rf₁ in Formula (AF) is a PFPE chain represented by Formula (7-1), and in Rf₁, h representing the average degree of polymerization represents 4.5, and i representing the average degree of polymerization represents 4.5) was obtained by performing the same operation as in Example 1 except that a compound represented by Formula (8-6) was used instead of the compound represented by Formula (8-1).

The compound represented by Formula (8-6) was synthesized by reacting a hydroxy group of N-(2-hydroxyethyl)-N-methylacetamide with epibromohydrin.

The structure of the obtained compound (AF) was identified by performing ¹H-NMR measurement and ¹⁹F-NMR measurement based on the following results.

¹H-NMR (acetone-D₆): δ [ppm]=2.00 (6H), 2.65 (6H), 3.20-3.80 (12H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 7

A compound represented by Formula (AG) was obtained by the following method.

4.05 g of a compound (AG) (Rf₁ in Formula (AG) is a PFPE chain represented by Formula (7-1), and in Rf₁, h representing the average degree of polymerization represents 4.5, and i representing the average degree of polymerization represents 4.5) was obtained by performing the same operation as in Example 1 except that a compound represented by Formula (8-7) was used instead of the compound represented by Formula (8-1).

The compound represented by Formula (8-7) was synthesized by reacting a hydroxy group of N-(2-hydroxyethyl)-N-ethylacetamide with epibromohydrin.

The structure of the obtained compound (AG) was identified by performing ¹H-NMR measurement and ¹⁹F-NMR measurement based on the following results.

¹H-NMR (acetone-D₆): δ [ppm]=1.65-1.85 (6H), 2.00 (6H), 3.00 (4H), 3.20-3.80 (12H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 8

A compound represented by Formula (AH) was obtained by the following method.

4.05 g of a compound (AH) (Rf₁ in Formula (AH) is a PFPE chain represented by Formula (7-1), and in Rf₁, h representing the average degree of polymerization represents 4.5, and i representing the average degree of polymerization represents 4.5) was obtained by performing the same operation as in Example 1 except that a compound represented by Formula (8-8) was used instead of the compound represented by Formula (8-1).

The compound represented by Formula (8-8) was synthesized by reacting a hydroxy group of N-(2-hydroxyethyl)pyrrolidone with epibromohydrin.

The structure of the obtained compound (AH) was identified by performing ¹H-NMR measurement and ¹⁹F-NMR measurement based on the following results.

¹H-NMR (acetone-D₆): δ [ppm]=2.00-2.65 (8H), 3.20-3.80 (16H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 9

A compound represented by Formula (AI) was obtained by the following method.

4.15 g of a compound (AI) (Rf₁ in Formula (AI) is a PFPE chain represented by Formula (7-1), and in Rf₁, h representing the average degree of polymerization represents 4.5, and i representing the average degree of polymerization represents 4.5) was obtained by performing the same operation as in Example 1 except that a compound represented by Formula (8-9) was used instead of the compound represented by Formula (8-1).

The compound represented by Formula (8-9) was synthesized by reacting a hydroxy group of N-(2-hydroxyethyl)piperidone with epibromohydrin.

The structure of the obtained compound (AI) was identified by performing ¹H-NMR measurement and ¹⁹F-NMR measurement based on the following results.

¹H-NMR (acetone-D₆): δ [ppm]=1.75-1.95 (4H), 2.00-2.65 (8H), 3.20-3.80 (16H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 10

A compound represented by Formula (AJ) was obtained by the following method.

4.00 g of a compound (AJ) (Rf₁ in Formula (AJ) is a PFPE chain represented by Formula (7-1), and in Rf₁, h representing the average degree of polymerization represents 4.5, and i representing the average degree of polymerization represents 4.5) was obtained by performing the same operation as in Example 1 except that a compound represented by Formula (8-10) was used instead of the compound represented by Formula (8-1).

The compound represented by Formula (8-10) was synthesized by reacting a hydroxy group of N-(2-hydroxyethyl)morpholinone with epibromohydrin.

The structure of the obtained compound (AJ) was identified by performing ¹H-NMR measurement and ¹⁹F-NMR measurement based on the following results.

¹H-NMR (acetone-D₆): δ [ppm]=1.75-1.95 (4H), 2.00-2.65 (8H), 3.20-3.80 (16H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 11

A compound represented by Formula (AK) was obtained by the following method.

A 100 mL eggplant flask was charged with 5 g of a compound represented by HOCH₂CF₂O(CF₂CF₂O)ₕ(CF₂O)ᵢCF₂CH₂OH (h representing the average degree of polymerization in the formula represent 4.5, and i representing the average degree of polymerization represent 4.5) (number-average molecular weight: 1000, molecular weight distribution: 1.1), 3.57 g of a compound represented by Formula (8-11), and 5 mL of t-butanol in a nitrogen gas atmosphere, and the mixture was stirred at room temperature until the mixture was uniform. 0.30 g of potassium tert-butoxide was added to the mixture, and the mixture was stirred at 70° C. for 16 hours to carry out a reaction.

The compound represented by Formula (8-11) was synthesized by the following method. Glycolylpiperidine and allyl glycidyl ether reacted with each other to obtain O-((3-allyloxy-(2-hydroxypropyl))glycolylpiperidine. Thereafter, the compound was synthesized by a method of protecting the hydroxy group of O-((3-allyloxy-(2-hydroxypropyl))glycolylpiperidine using dihydropyran and oxidizing the double bond using m-chloroperbenzoic acid (mCPBA).

The reaction solution obtained after the reaction was cooled to room temperature, 10 g of a 10% hydrogen chloride/methanol solution (hydrogen chloride-methanol reagent (5-10%) manufactured by Tokyo Chemical Industry Co., Ltd.) was added thereto, and the mixture was stirred at room temperature for 4 hours. Thereafter, the reaction solution was gradually transferred to a separatory funnel containing 25 mL of saturated aqueous sodium bicarbonate, and then extracted twice with 50 mL of ethyl acetate. The organic layer was washed with 25 mL of saline, 25 mL of saturated aqueous sodium bicarbonate, and 25 mL of saline in this order, and then dewatered with anhydrous sodium sulfate. The drying agent was separated by filtration, and the filtrate was concentrated, and the residues were purified by silica gel column chromatography, thereby obtaining 4.27 g of a compound (AK) (Rf₁ in Formula (AK) is a PFPE chain represented by Formula (7-1), and in Rf₁, h representing the average degree of polymerization represents 4.5, and i representing the average degree of polymerization represents 4.5).

The structure of the obtained compound (AK) was identified by performing ¹H-NMR measurement and ¹⁹F-NMR measurement based on the following results.

¹H-NMR (acetone-D₆): δ [ppm]=1.35-1.65 (12H), 3.40-3.95 (32H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 12

A compound represented by Formula (AL) was obtained by the following method.

4.51 g of a compound (AL) (Rf₁ in Formula (AL) is a PFPE chain represented by Formula (7-1), and in Rf₁, h representing the average degree of polymerization represents 4.5, and i representing the average degree of polymerization represents 4.5) was obtained by performing the same operation as in Example 11 except that a compound represented by Formula (8-12) was used instead of the compound represented by Formula (8-11).

The compound represented by Formula (8-12) was synthesized by the following method. N-Methyl-N-((3-allyloxy-(2-hydroxy)propan-1-oxy)ethyl)acetamide was obtained by reacting N-methyl-N-(hydroxyethyl)acetamide with allylglycidyl ether. Thereafter, the compound was synthesized by a method of protecting the hydroxy group of N-methyl-N-((3-allyloxy-(2-hydroxy)propan-1-oxy)ethyl)acetamide using dihydropyran and oxidizing the double bond using m-chloroperbenzoic acid (mCPBA).

The structure of the obtained compound (AL) was identified by performing ¹H-NMR measurement and ¹⁹F-NMR measurement based on the following results.

¹H-NMR (acetone-D₆): δ [ppm]=2.00 (6H), 2.70 (6H), 3.20-3.80 (28H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 13

A compound represented by Formula (BA) was obtained by the following method.

### (First Reaction)

A 100 mL eggplant flask was charged with 12.5 g of a compound represented by HOCH₂CF₂O(CF₂CF₂O)ₕ(CF₂O)ᵢCF₂CH₂OH (h representing the average degree of polymerization in the formula represent 4.5, and i representing the average degree of polymerization represent 4.5) (number-average molecular weight: 1,000, molecular weight distribution: 1.1), 2.40 g of a compound represented by Formula (8-4), and 12 mL of t-butanol in a nitrogen gas atmosphere, and the mixture was stirred at room temperature until the mixture was uniform. 1.10 g of potassium tert-butoxide was added to the mixture, and the mixture was stirred at 70° C. for 16 hours to carry out a reaction.

The reaction product obtained after the reaction was cooled to 25° C., transferred to a separatory funnel charged with 100 mL of water, and extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 5.62 g of a compound represented by Formula (9) as an intermediate compound 1.

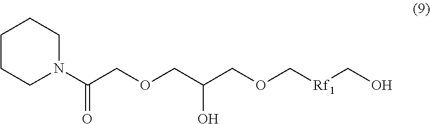

(Rf₁ in Formula (9) is a PFPE chain represented by Formula (7-1). In Rf₁, h representing the average degree of polymerization represents 4.5, and i representing the average degree of polymerization represents 4.5.)

### (Second Reaction)

Subsequently, a 100 mL eggplant flask was charged with 5.62 g of the compound represented by Formula (9), which was the intermediate compound 1 obtained above, 2.70 g of a compound represented by Formula (8-13), and 20 mL of t-butanol in a nitrogen gas atmosphere, and the mixture was stirred at room temperature until the mixture was uniform. 0.55 g of potassium tert-butoxide was added to the mixture, and the mixture was stirred at 70° C. for 16 hours to carry out a reaction.

The compound represented by Formula (8-13) was synthesized by a method of protecting a hydroxy group of ethylene glycol monoallyl ether using dihydropyran and then oxidizing a double bond using m-chloroperbenzoic acid (mCPBA).

The reaction solution obtained after the reaction was cooled to room temperature, 50 g of a 10% hydrogen chloride/methanol solution (hydrogen chloride-methanol reagent (5-10%), manufactured by Tokyo Chemical Industry Co., Ltd.) was added thereto, and the mixture was stirred at room temperature for 4 hours. Thereafter, the reaction solution was gradually transferred to a separatory funnel containing 100 mL of saturated aqueous sodium bicarbonate, and then extracted twice with 200 mL of ethyl acetate. The organic layer was washed with 100 mL of saline, 100 mL of saturated aqueous sodium bicarbonate, and 100 mL of saline in this order, and then dewatered with anhydrous sodium sulfate. The drying agent was separated by filtration, and the filtrate was concentrated, and the residues were purified by silica gel column chromatography, thereby obtaining 3.44 g of a compound (BA) (Rf₁ in Formula (BA) is a PFPE chain represented by Formula (7-1), and in Rf₁, h representing the average degree of polymerization represents 4.5, and i representing the average degree of polymerization represents 4.5.).

The structure of the obtained compound (BA) was identified by performing ¹H-NMR measurement and ¹⁹F-NMR measurement based on the following results.

¹H-NMR (acetone-D₆): δ [ppm]=1.55-1.75 (6H), 3.40-3.85 (22H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 14

A compound represented by Formula (BB) was obtained by the following method.

4.01 g of a compound (BB) (Rf₁ in Formula (BB) is a PFPE chain represented by Formula (7-1), and in Rf₁, h representing the average degree of polymerization represents 4.5, and i representing the average degree of polymerization represents 4.5) was obtained by performing the same operation as in Example 13 except that a compound represented by Formula (8-14) was used instead of the compound represented by Formula (8-13).

The compound represented by Formula (8-14) was synthesized by the following method. A double bond of the ethylene glycol monoallyl ether was oxidized using m-chloroperbenzoic acid (mCPBA), and reacted with a hydroxy group of glycidol. After the reaction, two hydroxy groups of the obtained compound were synthesized by a method of protecting the two hydroxy groups with dihydropyran.

The structure of the obtained compound (BB) was identified by performing ¹H-NMR measurement and ¹⁹F-NMR measurement based on the following results.

¹H-NMR (acetone-D₆): δ [ppm]=1.55-1.75 (6H), 3.40-3.85 (28H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 15

A compound represented by Formula (BC) was obtained by the following method.

4.11 g of a compound (BC) (Rf₁ in Formula (BC) is a PFPE chain represented by Formula (7-1), and in Rf₁, h representing the average degree of polymerization represents 4.5, and i representing the average degree of polymerization represents 4.5) was obtained by performing the same operation as in Example 13 except that a compound represented by Formula (8-6) was used instead of the compound represented by Formula (8-4).

The structure of the obtained compound (BC) was identified by performing ¹H-NMR measurement and ¹⁹F-NMR measurement based on the following results.

¹H-NMR (acetone-D₆): δ [ppm]=2.00 (3H), 2.65 (3H), 3.20-3.80 (12H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 16

A compound represented by Formula (BD) was obtained by the following method.

4.31 g of a compound (BD) (Rf₁ in Formula (BD) is a PFPE chain represented by Formula (7-1), and in Rf₁, h representing the average degree of polymerization represents 4.5, and i representing the average degree of polymerization represents 4.5) was obtained by performing the same operation as in Example 13 except that a compound represented by Formula (8-6) was used instead of the compound represented by Formula (8-13).

The structure of the obtained compound (BD) was identified by performing ¹H-NMR measurement and ¹⁹F-NMR measurement based on the following results.

¹H-NMR (acetone-D₆): δ [ppm]=1.65-1.85 (4H), 3.40-3.85 (26H), 3.85-4.10 (4H)

¹⁹F-NMR (acetone-D₆): δ [ppm]=−55.5 to −51.5 (9F), −78.5 (2F), −80.5 (2F), −91.0 to −88.5 (18F)

### Example 17

A compound represented by Formula (BE) was obtained by the following method.

4.50 g of a compound (BE) (Rf₂ in Formula (BE) is a PFPE chain represented by Formula (7-2), and in Rf₂, j representing the average degree of polymerization represents 4.5) was obtained by performing the same operation as in Example 4 except that a compound represented by HOCH₂CF₂CF₂O(CF₂CF₂CF₂O)jCF₂CF₂CH₂OH (representing the average degree of polymerization in the formula represent 4.5) (number-average molecular weight: 1,000, molecular weight distribution: 1.1) was used instead of the compound represented by HOCH₂CF₂O(CF₂CF₂O)ₕ(CF₂O)ᵢCF₂CH₂OH.

The structure of the obtained compound (BE) was identified by performing ¹H-NMR measurement and ¹⁹F-NMR measurement based on the following results.

¹H-NMR (acetone-D₆): δ [ppm]=1.30-1.45 (4H), 1.95-2.10 (8H), 3.20-3.65 (12H), 3.65-3.95 (4H), 3.75-4.00 (2H), 3.85-4.10 (4H)

¹⁹F-NMR (acetone-D₆): δ [ppm]=−84.0 to −83.0 (18F), −86.4 (4F), −124.3 (4F), −130.0 to −129.0 (9F)

### Example 18

A compound represented by Formula (BF) was obtained by the following method.

4.44 g of a compound (BF) (Rf₃ in Formula (BE) is a PFPE chain represented by Formula (7-3), and in Rf₃, k representing the average degree of polymerization represents 3.0) was obtained by performing the same operation as in Example 4 except that a compound represented by HOCH₂CF₂CF₂CF₂O(CF₂CF₂CF₂CF₂O)ₖCF₂CF₂CF₂CH₂OH (k representing the average degree of polymerization in the formula represent 3.0) (number-average molecular weight: 1,000, molecular weight distribution: 1.1) was used instead of the compound represented by HOCH₂CF₂O(CF₂CF₂O)ₕ(CF₂O)ᵢCF₂CH₂OH.

The structure of the obtained compound (BF) was identified by performing ¹H-NMR measurement and ¹⁹F-NMR measurement based on the following results.

¹H-NMR (acetone-D₆): δ [ppm]=1.30-1.45 (4H), 1.95-2.10 (8H), 3.20-3.65 (12H), 3.65-3.95 (4H), 3.75-4.00 (2H), 3.85-4.10 (4H)

¹⁹F-NMR (acetone-D₆): δ [ppm]=−84.0 to −83.0 (16F), −122.5 (4F), −126.0 (12F), −129.0 to −128.0 (4F)

The structures of R¹, R², R³, R⁴, and R⁵ in a case where the compounds (AA) to (AL), and (BA) to (BF) of Examples 1 to 18 obtained as described above were each applied to Formula (1) are listed in Table 1.

TABLE 1

| Compound | R¹ | R² | R³ | R⁴ | R⁵ |
|---|---|---|---|---|---|
| Compound | (3) | (4) | (7-1) | (4) | (3) |
| (AA) | X²═—CH₂— | n = m = l = 1 | | n = m = l = 1 | X²═—CH₂— |
| | A=B═—CH₃ | E = single | | E = single | A═B═—CH₃ |
| | | bond | | bond | |
| Compound | (3) | (4) | (7-1) | (4) | (3) |
| (AB) | X²═—CH₂— | n = m = l = 1 | | n = m = l = 1 | X²═—CH₂— |
| | A═B═—CH₂CH₃ | E = single | | E = single | A═B═—CH₂CH₃ |
| | | bond | | bond | |
| Compound | (3) | (4) | (7-1) | (4) | (3) |
| (AC) | X²═—CH₂— | n = m = l = 1 | | n = m = l = 1 | X²═—CH₂— |
| | —A—B—═—(CH₂)₄— | E = single | | E = single | —A—B—═—(CH₂)₄— |
| | | bond | | bond | |
| Compound | (3) | (4) | (7-1) | (4) | (3) |
| (AD) | X²═—CH₂— | n = m = l = 1 | | n = m = l = 1 | X²═—CH₂— |
| | —A—B—═—(CH₂)₅— | E = single | | E = single | —A—B—═—(CH₂)₅— |
| | | bond | | bond | |
| Compound | (3) | (4) | (7-1) | (4) | (3) |
| (AE) | X²═—CH₂— | n = m = l = 1 | | n = m = l = 1 | X²═—CH₂— |
| | —A—B—═—(CH₂)₂—O—(CH₂)₂— | E = single | | E = single | —A—B—═—(CH₂)₂—O—(CH₂)₂— |
| | | bond | | bond | |
| Compound | (2) | (4) | (7-1) | (4) | (2) |
| (AF) | X¹═—CH₂CH₂— | n = m = l = 1 | | n = m = 1 = 1 | X¹═—CH₂CH₂— |
| | Y═Z═—CH₃ | E = single | | E = single | Y═Z═—CH₃ |
| | | bond | | bond | |

TABLE 2

| Compound | R¹ | R² | R³ | R⁴ | R⁵ |
|---|---|---|---|---|---|
| Compound | (2) | (4) | (7-1) | (4) | (2) |
| (AG) | X¹═— CH₂CH₂—Y═— CH₂CH₃ | n = m = l = 1 | | n = m = l = 1 | X¹═—CH₂CH₂— |
| | Z═—CH₃ | E = single | | E = single | Y═—CH₂CH₃ |
| | | bond | | bond | Z═—CH₃ |
| Compound | (2) | (4) | (7-1) | (4) | (2) |
| (AH) | X¹═—CH₂CH₂— | n = m = l = 1 | | n = m = l = 1 | X¹═—CH₂CH₂— |
| | —Y—Z—═—(CH₂)₃— | E = single | | E = single | —Y—Z—═—(CH₂)₃— |
| | | bond | | bond | |
| Compound | (2) | (4) | (7-1) | (4) | (2) |
| (AI) | X¹═—CH₂CH₂— | n = m = l = 1 | | n = m = l = 1 | X¹═—CH₂CH₂— |
| | —Y—Z—═—(CH₂)₄— | E = single | | E = single | —Y—Z—═—(CH₂)₄— |
| | | bond | | bond | |
| Compound | (2) | (4) | (7-1) | (4) | (2) |
| (AJ) | X¹═—CH₂CH₂— | n = m = l = 1 | | n = m = l = 1 | X¹═—CH₂CH₂— |
| | —Y—Z—═—(CH₂)₂—O—CH₂— | E = single | | E = single | —Y—Z—═—(CH₂)₂—O—CH₂— |
| | | bond | | bond | |
| Compound | (3) | (4) | (7-1) | (4) | (3) |
| (AK) | X²═—CH₂— | n = m = l | | n = m = l | X²═—CH₂— |
| | —A—B—═—(CH₂)₅— | 1 = 2 | | 1 = 2 | —A—B—═—(CH₂)₅— |
| | | E = single | | E = single | |
| | | bond | | bond | |
| Compound | (2) | (4) | (7-1) | (4) | (2) |
| (AL) | X¹═—CH₂CH₂— | n = m = l | | n = m = l | X¹═—CH₂CH₂— |
| | Y═Z═—CH₃ | 1 = 2 | | 1 = 2 | Y═Z═—CH₃ |
| | | E = single | | E = single | |
| | | bond | | bond | |

TABLE 3

| Compound | R¹ | R² | R³ | R⁴ | R⁵ |
|---|---|---|---|---|---|
| Compound | (3) | (4) | (7-1) | (4) | Hydrogen atom |
| (BA) | X²═—CH₂— | n = m = l = 1 | | n = m = l = 1 | |
| | —A—B—═—(CH₂)₅— | E = single | | E = —CH₂CH₂O— | |
| | | bond | | | |
| Compound | (3) | (4) | (7-1) | (4) | Hydrogen atom |
| (BB) | X²═—CH₂— | n = m = l = 1 | | n = m = l | |
| | —A—B—═—(CH₂)₅— | E = single | | 1 = 2 | |
| | | bond | | E = —CH₂CH₂O— | |
| Compound | (2) | (4) | (7-1) | (4) | Hydrogen atom |
| (BC) | X¹ ═—CH₂CH₂— | n = m = l = 1 | | n = m = l = 1 | |
| | Y═Z═—CH₃ | E = single | | E = —CH₂CH₂O— | |
| | | bond | | | |
| Compound | (2) | (4) | (7-1) | (4) | (3) |
| (BD) | X¹═—CH₂CH₂— | n = m = l = 1 | | n = m = l = 1 | X²═—CH₂— |
| | Y═Z═—CH₃ | E = single | | E = single bond | —A—B—═—(CH₂)₅— |
| | | bond | | | |
| Compound | (3) | (4) | (7-2) | (4) | (3) |
| (BE) | X²═—CH₂— | n = m = l = 1 | | n = m = l = 1 | X²═—CH₂— |
| | —A—B—═—(CH₂)₅— | E = single | | E = single bond | —A—B—═—(CH₂)₅— |
| | | bond | | | |
| Compound | (3) | (4) | (7-3) | (4) | (3) |
| (BF) | X²═—CH₂— | n = m = l = 1 | | n = m = l = 1 | X²═—CH₂— |
| | —A—B—═—(CH₂)₅— | E = single | | E = single bond | —A—B—═—(CH₂)₅— |
| | | bond | | | |

### Comparative Example 1

A compound represented by Formula (ZA) was synthesized by the method described in Patent Document 1.

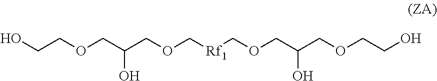

(Rf₁ in Formula (ZA) is a PFPE chain represented by Formula (7-1) described above. In Rf₁, h representing the average degree of polymerization represents 4.5, and i representing the average degree of polymerization represents 4.5.)

### Comparative Example 2

A compound represented by Formula (ZB) was synthesized with reference to the method described in Patent Document 2. In Example 1 of Patent Document 2, glycidyl 4-acetaminophenyl ether was used instead of glycidyl phenyl ether.

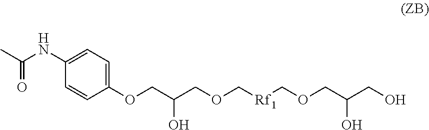

(Rf₁ in Formula (ZB) is a PFPE chain represented by Formula (7-1) described above. In Rf₁, h representing the average degree of polymerization represents 4.5, and i representing the average degree of polymerization represents 4.5.)

### Comparative Example 3

A compound represented by Formula (ZC) was synthesized with reference to the method described in Patent Document 4. In Example 1 of Patent Document 4, 2-hydroxyacetamide was used instead of 3-hydroxypropionamide.

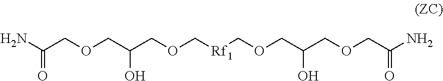

(Rf₁ in Formula (ZC) is a PFPE chain represented by Formula (7-1). In Rf₁, h representing the average degree of polymerization represents 4.5, and i representing the average degree of polymerization represents 4.5.)

### Comparative Example 4

A compound represented by Formula (ZD) was synthesized by the method described in Patent Document 4.

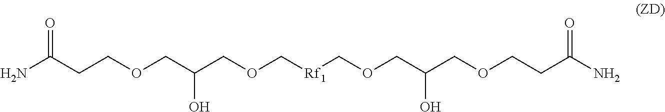

(Rf₁ in Formula (ZD) is a PFPE chain represented by Formula (7-1). In Rf₁, h representing the average degree of polymerization represents 4.5, and i representing the average degree of polymerization represents 4.5.)

### Comparative Example 5

A compound represented by Formula (ZE) was synthesized by the method described in Patent Document 4.

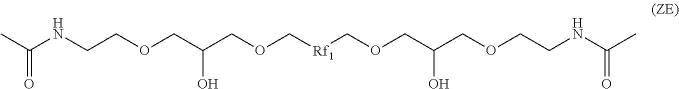

(Rf₁ in Formula (ZE) is a PFPE chain represented by Formula (7-1) described above. In Rf₁, h representing the average degree of polymerization represents 4.5, and i representing the average degree of polymerization represents 4.5.)

The number-average molecular weights (Mn) of the compounds of Examples 1 to 18 and Comparative Examples 1 to 5 obtained above were measured by the above-described method. The results are listed in Table 4.

Next, a lubricating layer forming solution was prepared using the compounds obtained in Examples 1 to 18 and Comparative Examples 1 to 5 by the following method. Further, a lubricating layer of a magnetic recording medium was formed by the following method using the obtained lubricating layer forming solution, thereby obtaining magnetic recording media of Examples 1 to 18 and Comparative Examples 1 to 5.

### “Solution for Forming Lubricating Layer”

The compounds obtained in Examples 1 to 18 and Comparative Examples 1 to 5 were each dissolved in VERTREL (registered trademark) XF (trade name, manufactured by DuPont Mitsui Fluorochemicals Co., Ltd.), which is a fluorine-based solvent, and diluted with VERTREL XF such that the film thickness in a case where the solution was applied onto the protective layer reached 9.0 A° to 9.5 A°, thereby preparing a lubricating layer forming solution.

### “Magnetic Recording Medium”

A magnetic recording medium in which an adhesive layer, a soft magnetic layer, a first underlayer, a second underlayer, a magnetic layer, and a protective layer were sequentially provided on a substrate having a diameter of 65 mm was prepared. The protective layer was formed of carbon.

The protective layer of the magnetic recording medium on which each layer up to the protective layer was formed was coated with each lubricating layer forming solution of Examples 1 to 18 and Comparative Examples 1 to 5 by a dipping method. Further, the dipping method was carried out under the conditions of an immersion speed of 10 mm/sec, an immersion time of 30 see, and a pulling-up speed of 1.2 mm/sec.

Thereafter, the magnetic recording medium coated with the lubricating layer forming solution was placed in a constant temperature tank, and a thermal treatment was performed at 120° C. for 10 minutes to remove the solvent in the lubricating layer forming solution and to improve the adhesion between the protective layer and the lubricating layer so that a lubricating layer was formed on the protective layer, thereby obtaining a magnetic recording medium.

### (Measurement of Film Thickness)

The film thicknesses of the lubricating layers of the magnetic recording media of Examples 1 to 18 and Comparative Examples 1 to 5 obtained above were measured using FT-IR (trade name: Nicolet iS50, manufactured by Thermo Fisher Scientific). The results are listed in Table 4.

Next, ae wear resistance test, a chemical substance resistance test, and a corrosion resistance test described below were performed on the magnetic recording media of Examples 1 to 18 and Comparative Examples 1 to 5.

### (Wear Resistance Test)

Using a pin-on-disk type friction and wear tester, an alumina ball having a diameter of 2 mm as a contactor was slid on the lubricating layer of the magnetic recording medium under a load of 40 gf and a sliding speed of 0.25 m/sec, and a friction coefficient of the surface of the lubricating layer was measured. Then, a sliding time until the friction coefficient of the surface of the lubricating layer was rapidly increased was measured. A sliding time until the friction coefficient was rapidly increased was measured four times for each lubricating layer of each magnetic recording medium, and an average value (time) thereof was used as an index of the wear resistance of the lubricant coating film.

The results of the magnetic recording medium using the compounds of Examples 1 to 18 and the compounds of Comparative Examples 1 to 5 are listed in Table 4. The evaluation criteria of the wear resistance according to the sliding time until the friction coefficient was rapidly increased are as follows.

### “Evaluation Criteria”

A: 650 sec or greater
B: 550 sec or greater and less than 650 sec
C: 450 sec or greater and less than 550 sec
D: less than 450 sec

The time until the friction coefficient is rapidly increased can be used as an index of wear resistance of the lubricating layer for the following reasons. The reason is that, in a case where the abrasion progresses by using the magnetic recording medium and the lubricating layer is lost due to the abrasion, the protector and the protective layer are in direct contact with each other, and thus the friction coefficient is rapidly increased. It is considered that the time until the friction coefficient is rapidly increased is correlated with the friction test.

### (Chemical Substance Resistance Test)

Contamination of the magnetic recording medium due to an environmental substance generating a contaminant in a high-temperature environment was examined by the following method. A Si ion was used as the environmental substance, and a Si adsorption amount was measured as the amount of the contaminant which contaminated the magnetic recording medium generated by the environmental substance.

Specifically, the magnetic recording medium to be evaluated was held in a high-temperature environment of a temperature of 85° C. and a humidity of 0% for 240 hours in the presence of siloxane-based Si gum. Next, the adsorption amount of Si present on the surface of the magnetic recording medium was analyzed and measured by secondary ion mass spectrometry (SIMS), and the degree of contamination with Si ions was evaluated as the Si adsorption amount. The Si adsorption amount was evaluated based on the following evaluation criteria using a numerical value in a case where the result of Comparative Example 1 was set to 1.00. The results are listed in Table 4.

### “Evaluation Criteria”

A: The Si adsorption amount was less than 0.70.
B: The Si adsorption amount was 0.70 or greater and less than 0.90.
C: The Si adsorption amount was 0.90 or greater and less than 1.10.
D: The Si adsorption amount was 1.10 or greater.

### (Corrosion Resistance Test)

The magnetic recording medium was exposed to an environment of a temperature of 85° C. and a relative humidity of 90% for 48 hours. Thereafter, the number of the corrosion spots having a diameter of 5 microns or more, which had occurred on the surface of the magnetic recording medium, was counted using an optical surface analyzer (Candela 7140 manufactured by KLA-Tencor Corporation), and evaluated based on the following evaluation criteria. The results are listed in Table 4.

### “Evaluation Criteria”

A: less than 150 sites
B: 150 sites or greater and less than 250 sites
C: 250 sites or greater and less than 1,000 sites
D: 1,000 sites or greater

TABLE 4

| | | Molecular | Film thickness | Abrasion | Chemical | Corrosion |
|---|---|---|---|---|---|---|
| | Compound | weight | (Å) | resistance | resistance | resistance |
| Example 1 | Compound (AA) | 1352 | 9.0 | A | B | B |
| Example 2 | Compound (AB) | 1408 | 9.1 | A | B | B |
| Example 3 | Compound (AC) | 1402 | 9.3 | B | B | A |
| Example 4 | Compound (AD) | 1410 | 9.1 | B | B | A |
| Example 5 | Compound (AE) | 1409 | 9.2 | A | B | B |
| Example 6 | Compound (AF) | 1333 | 9.4 | A | B | B |
| Example 7 | Compound (AG) | 1258 | 9.2 | A | B | B |
| Example 8 | Compound (AH) | 1284 | 9.0 | B | B | A |
| Example 9 | Compound (AI) | 1282 | 9.0 | B | B | A |
| Example 10 | Compound (AJ) | 1314 | 9.3 | B | B | B |
| Example 11 | Compound (AK) | 1544 | 9.1 | A | B | B |
| Example 12 | Compound (AL) | 1498 | 9.0 | A | B | B |
| Example 13 | Compound (BA) | 1317 | 9.0 | A | B | A |
| Example 14 | Compound (BB) | 1391 | 9.3 | A | B | B |
| Example 15 | Compound (BC) | 1246 | 9.1 | A | B | A |
| Example 16 | Compound (BD) | 1328 | 9.2 | B | A | A |
| Example 17 | Compound (BE) | 1410 | 9.2 | B | B | A |
| Example 18 | Compound (BF) | 1410 | 9.0 | B | B | A |
| Comparative | Compound (ZA) | 1199 | 9.2 | C | C | C |
| Example 1 | | | | | | |
| Comparative | Compound (ZB) | 1268 | 9.3 | B | A | C |
| Example 2 | | | | | | |
| Comparative | Compound (ZC) | 1197 | 9.1 | C | C | C |
| Example 3 | | | | | | |
| Comparative | Compound (ZD) | 1198 | 9.4 | C | C | C |
| Example 4 | | | | | | |
| Comparative | Compound (ZE) | 1200 | 9.1 | B | B | C |
| Example 5 | | | | | | |

As shown in Table 4, in all of the magnetic recording media of Examples 1 to 18, the evaluation was A or B in all the evaluation items. Therefore, it was confirmed that the lubricating layer of the magnetic recording medium of Examples 1 to 18 had excellent wear resistance and chemical substance resistance, and had a high effect of suppressing corrosion of the magnetic recording medium.

On the other hand, in Comparative Example 1, —CH₂CH₂OH was disposed at both ends, and a compound (ZA) not including the end group having N,N-substituted amide was used. Therefore, in Comparative Example 1, it is presumed that the adhesion of the lubricating layer to the protective layer is too strong, so that the wear resistance is insufficient, and the fat-solubility of the lubricating layer is insufficient, and thus the environment substances that generate the contaminant and the water, which causes the corrosion of the magnetic recording medium, are attracted to the lubricating layer, and thus the corrosion resistance and the chemical substance resistance are not sufficiently obtained. As a result, in the magnetic recording medium of Comparative Example 1 in which the compound (ZA) was used, the evaluation was C in all the evaluation items.

In addition, in Comparative Example 2, a compound (ZB) in which an amide group (—NHC(═O)CH₃) in which one hydrogen atom is bonded to a nitrogen atom constituting an amide bond through a phenylene group is disposed at one end is used. In Comparative Example 2, since the compound (ZB) not including the end group having an N,N-substituted amide was used, the evaluation of the corrosion resistance is presumed to be C because the fat-solubility of the end group was insufficient, and the evaluation of the wear resistance and the chemical substance resistance was satisfactory.

In addition, in both of Comparative Example 3 and Comparative Example 4, a compound (compound (ZC) and (ZD)) was used in which an amide group disposed at both ends was a group in which two hydrogen atoms were bonded to a nitrogen atom constituting an amide bond, and the compound did not include an end group having an N,N-substituted amide. Therefore, in Comparative Example 3 and Comparative Example 4, it is presumed that the fat-solubility of the compounds (ZC) and (ZD) was insufficient. In addition, in Comparative Example 3 and Comparative Example 4, it is presumed that the amide groups disposed at both ends of the compound aggregate or interact with the hydroxy group in the compound to generate a hydroxy group which does not participate in the bonding with the active point on the protective layer, and thus the environment substances that generate the contaminant and the water that causes the corrosion of the magnetic recording medium are attracted to the lubricating layer. From these results, it is presumed that the evaluation was C in all the evaluation items in Comparative Example 3 using the compound (ZC) and Comparative Example 4 using the compound (ZD).

In addition, in Comparative Example 5, a compound (ZE) in which an amide group disposed at both ends is a group in which one hydrogen atom is bonded to a nitrogen atom constituting an amide bond is used. Therefore, in Comparative Example 5, the wear resistance and the chemical substance resistance were improved as compared with Comparative Examples 3 and 4 in which a compound in which two hydrogen atoms were bonded to a nitrogen atom constituting an amide bond was used. However, in Comparative Example 5, it is presumed that the evaluation of the corrosion resistance is C because the fat-solubility of the compound (ZE) is insufficient.

### INDUSTRIAL APPLICABILITY

A lubricating layer having excellent adhesion, satisfactory chemical substance resistance and wear resistance, and a high corrosion suppression effect of the magnetic recording medium can be formed even in a case where the thickness thereof is small by using the lubricant for a magnetic recording medium containing the fluorine-containing ether compound of the present invention.

### REFERENCE SIGNS LIST

10: magnetic recording medium
11: substrate
12: adhesive layer
13: soft magnetic layer
14: first underlayer
15: second underlayer
16: magnetic layer
17: protective layer
18: lubricating layer

1: A fluorine-containing ether compound which is represented by Formula (1),
R¹—O—R²—CH₂—R³—CH₂—R⁴—O—R⁵ (1)
(in Formula (1), R³ represents a perfluoropolyether chain; R² and R⁴ represent a divalent linking group having one or more polar groups, and may be the same as or different from each other; R¹ and R⁵ represent an end group represented by Formula (2), an end group represented by Formula (3), or a hydrogen atom, where R¹ and R⁵ may be the same as or different from each other; and at least one of R¹ or R⁵ represents the end group represented by Formula (2) or (3)), 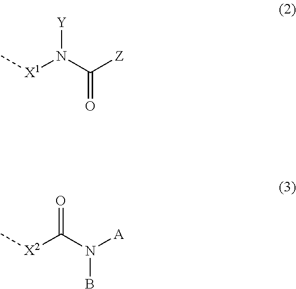
(in Formula (2), X¹ represents an alkylene group having 1 to 30 carbon atoms; Y and Z each independently represent an aliphatic group having 1 to 30 carbon atoms, which may have a polar group or an ether oxygen atom; and Y and Z may be bonded to each other to form a cyclic structure),
(in Formula (3), X² represents an alkylene group having 1 to 30 carbon atoms; A and B each independently represent an aliphatic group having 1 to 30 carbon atoms, which may have a polar group or an ether oxygen atom; and A and B may be bonded to each other to form a cyclic structure).

2: The fluorine-containing ether compound according to claim 1,
wherein R¹ and R⁵ in Formula (1) each independently represent the end group represented by Formula (2) or (3).

3: The fluorine-containing ether compound according to claim 2,
wherein R¹ and R⁵ in Formula (1) are the same as each other.

4: The fluorine-containing ether compound according to claim 2,
wherein R¹ and R⁵ in Formula (1) are different from each other.

5: The fluorine-containing ether compound according to claim 1,
wherein one of R¹ or R⁵ in Formula (1) represents the end group represented by Formula (2) or (3), and the other represents a hydrogen atom.

6: The fluorine-containing ether compound according to claim 1,
wherein —R²—O— and —R⁴—O— in Formula (1) are each independently represented by Formula (4), 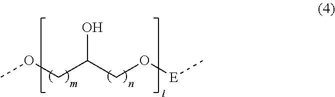
(in Formula (4), 1 represents an integer of 1 to 3; 1 pieces of m's each independently represent an integer of 1 to 6; 1 pieces of n's each independently represent an integer of 1 to 6; at least one of m or n in one repeating unit represents 1; E represents a single bond, —CH₂CH₂O— (a leftmost carbon atom is bonded to an oxygen atom in a repeating unit), —CH₂CH₂CH₂O— (a leftmost carbon atom is bonded to an oxygen atom in a repeating unit), or —CH₂CH₂CH₂CH₂O— (a leftmost carbon atom is bonded to an oxygen atom in a repeating unit); and in Formula (4), a leftmost oxygen atom is bonded to a methylene group bonded to R³, and E is bonded to R¹ or R⁵).

7: The fluorine-containing ether compound according to claim 1,
wherein —R²—O— and —R⁴—O— in Formula (1) are each independently represented by Formula (5-1) or (5-2), 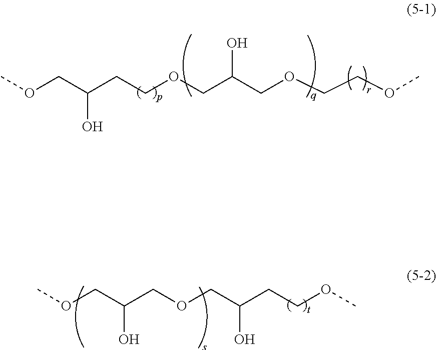
(in Formula (5-1), p represents an integer of 0 to 3, q represents an integer of 0 to 2, and r represents an integer of 1 to 3; and in Formula (5-1), a leftmost oxygen atom is bonded to a methylene group bonded to R³, and a rightmost oxygen atom is bonded to R¹ or R⁵),
(in Formula (5-2), s represents an integer of 0 to 2, and t represents an integer of 0 to 3; and in Formula (5-2), a leftmost oxygen atom is bonded to the methylene group bonded to R³, and a rightmost oxygen atom is bonded to R¹ or R⁵).

8: The fluorine-containing ether compound according to claim 1,
wherein R³ in Formula (1) represents a perfluoropolyether chain represented by Formula (6),
—(CF₂)_{w1}—O—(CF₂O)_{w2}—(CF₂CF₂O)_{w3}—(CF₂CF₂CF₂O)_{w4}—(CF₂CF₂CF₂CF₂O)_{w5}—(CF₂)_{w6}— (6)
(in Formula (6), w2, w3, w4, and w5 represent an average degree of polymerization, and each independently represent 0 to 20, where all of w2, w3, w4, and w5 do not represent 0 at the same time; w1 and w6 represent an average value representing the number of CF₂'s, and each independently represent 1 to 3; and an arrangement order of (CF₂O), (CF₂CF₂O), (CF₂CF₂CF₂O), and (CF₂CF₂CF₂CF₂O), which are repeating units in Formula (6), is not particularly limited).

9: The fluorine-containing ether compound according to claim 1,
wherein R³ in Formula (1) represents any one selected from the group consisting of perfluoropolyether chains represented by any one of Formulae (7-1) to (7-4),
—CF₂—(OCF₂CF₂)ₕ—(OCF₂)ᵢ—OCF₂— (7-1)
(in Formula (7-1), h and i represent an average degree of polymerization, where h represents 1 to 20 and i represents 0 to 20),
—CF₂CF₂—(OCF₂CF₂CF₂)ⱼ—OCF₂CF₂— (7-2)
(in Formula (7-2), j represents an average degree of polymerization, and represents 1 to 15),
—CF₂CF₂CF₂—(OCF₂CF₂CF₂CF₂)ₖ—OCF₂CF₂CF₂— (7-3)
(in Formula (7-3), k represents an average degree of polymerization, and represents 1 to 10),
—(CF₂)_{w7}—O—(CF₂CF₂CF₂O)_{w8}—(CF₂CF₂O)_{w9}—(CF₂)_{w10}— (7-4)
(in Formula (7-4), w8 and w9 represent an average degree of polymerization, and each independently represents 1 to 20; and w7 and w10 represent an average value representing the number of CF₂'s, and each independently represent 1 to 2).

10: The fluorine-containing ether compound according to claim 1,
wherein the fluorine-containing ether compound has a number-average molecular weight of 500 to 10000.

11: A lubricant for a magnetic recording medium, comprising:
the fluorine-containing ether compound according to claim 1.

12: A magnetic recording medium, which is provided with at least a magnetic layer, a protective layer, and a lubricating layer in this order on a substrate,
wherein the lubricating layer contains the fluorine-containing ether compound according to claim 1.

13: The magnetic recording medium according to claim 12,
wherein the lubricating layer has an average film thickness of 0.5 nm to 2.0 nm.
